(19) United States
(12) Patent Application Publication
(10) Pub. No.: US 2025/0000774 A1
(43) Pub. Date: Jan. 2, 2025

(54) STEM CELL STIMULATING COMPOSITIONS AND METHODS

(71) Applicant: Medicell Technologies LLC, Carlsbad, CA (US)

(72) Inventor: Nikolay Turovets, Carlsbad, CA (US)

(21) Appl. No.: 18/754,637

(22) Filed: Jun. 26, 2024

Related U.S. Application Data
(60) Provisional application No. 63/510,499, filed on Jun. 27, 2023.

(51) Int. Cl. A61K 8/64; A61K 8/31; A61K 8/35; A61K 8/44; A61K 8/46; A61K 8/67; A61K 8/9711; A61K 8/9717; A61K 8/9789; A61Q 3/00; A61Q 19/00; A61Q 19/08

(52) U.S. Cl. CPC A61K 8/64; A61K 8/31; A61K 8/355; A61K 8/442; A61K 8/46; A61K 8/673; A61K 8/675; A61K 8/678; A61K 8/9711; A61K 8/9717; A61K 8/9789; A61Q 3/00; A61Q 19/001; A61Q 19/08; A61K 2800/85

(57) ABSTRACT

Disclosed herein are compositions and methods of using a topical formulation on lips and nails. The topical formulation might comprise one for more defensins. The topical formulation might also comprise other cosmetically acceptable ingredients. The topical formulation might be used to alleviate a cosmetic concern of the lips or nails.

### CROSS-REFERENCE TO RELATED APPLICATIONS

This application claims the benefit of U.S. Provisional Application No. 63/510,499, filed on Jun. 27, 2023, which is incorporated by reference herein in its entirety for all purposes.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

The present disclosure provides compositions and methods for alleviating cosmetic concerns of an individual. Some aspects of the present disclosure provide compositions for topical formulations and methods of use for alleviating a cosmetic concern of an individual.

In one aspect, the present disclosure provides a composition comprising a topical formulation formulated for use on a lip or a nail that comprises: a) a first defensin, and b) a cosmetically acceptable ingredient.

In some embodiments, the topical formulation is formulated for use on the lip and the topical formulation is capable of alleviating a cosmetic concern of the lip. In some embodiments, the cosmetic concern of the lip comprises wrinkles on the lip, wrinkles around contour of the lip, an appearance of aging of the lip, discoloration of the lip, dryness of the lip, or any combinations thereof.

In some embodiments, the topical formulation is formulated for use on the nail and the topical formulation is capable of alleviating a cosmetic concern of the nail or stimulating a regeneration of the nail. In some embodiments, the nail comprises a nail portion selected from the group consisting of nail root, eponychium, proximal nail fold, lanula, cuticle, lateral nail fold, nail body/plate and hyponychium. In some embodiments, the cosmetic concern of the nail comprises brittle nail, thin nail, weakness of nail, nail discoloration, slow nail growth, nail surface irregularities, nail surface smoothness, nail vertical ridges, nail horizontal ridges, nail hydration, cuticle hydration, aesthetic appearance of the nail, aesthetic appearance of the cuticle, or any combinations thereof.

In some embodiments of any of the compositions of the present disclosure, the first defensin is selected from the group consisting of alpha-defensin 1, alpha-defensin 5, alpha-defensin 6, neutrophil defensin 1, neutrophil defensin 2, neutrophil defensin 3, neutrophil defensin 4, theta-defensin, beta-defensin 1, beta-defensin 2, beta-defensin 3 and beta-defensin 4. In some embodiments of any of the compositions of the present disclosure, the first defensin is present in a concentration effective to recruit Lgr6+ stem cells. In some embodiments of any of the compositions of the present disclosure, the first defensin is present in a concentration of about 0.01 ng/ml to about 1000 ng/ml. In some embodiments of any of the compositions of the present disclosure, the first defensin is present in a concentration of about 0.01 ng/ml to about 100 ng/ml. In some embodiments of any of the compositions of the present disclosure, the first defensin is present in a concentration of about 1 ng/ml to about 100 ng/ml.

In some embodiments of any of the compositions of the present disclosure, the topical formulation further comprises a second defensin, wherein the second defensin is selected from the group consisting of alpha-defensin 1, alpha-defensin 5, alpha-defensin 6, neutrophil defensin 1, neutrophil defensin 2, neutrophil defensin 3, neutrophil defensin 4, theta-defensin, beta-defensin 1, beta-defensin 2, beta-defensin 3 and beta-defensin 4. In some embodiments, the second defensin is present in a concentration effective to recruit Lgr6+ stem cells. In some embodiments, the second defensin is present in a concentration of about 0.01 ng/ml to about 1000 ng/ml. In some embodiments, the second defensin is present in a concentration of about 0.01 ng/ml to about 100 ng/ml. In some embodiments, the second defensin is present in a concentration of about 1 ng/ml to about 100 ng/ml. In some embodiments, the first defensin and the second defensin are present in a mass ratio of about 1:10 to about 10:1. In some embodiments, the first defensin and the second defensin are present in a mass ratio of about 1:5 to about 5:1.

In some embodiments of any of the compositions of the present disclosure, the cosmetically acceptable ingredient is selected from the group consisting of Leuconostoc/radish root ferment filtrate, albumin, l-alanyl-l-glutamine, phospholipids, tocopheryl acetate, ubiquinone, Caesalpinia spinosa fruit extract, Helianthus annuus sprout extract, Pistacia lentiscus gum, squalane, panthenol, biotin, niacinamide, d-l methionine and any combinations thereof. In some embodiments of any of the compositions of the present disclosure, the cosmetically acceptable ingredient is selected from the group consisting of Leuconostoc/radish root ferment filtrate, albumin, l-alanyl-l-glutamine, phospholipids, tocopheryl acetate, ubiquinone, Undaria pinnatifida extract, Caesalpinia spinosa fruit extract, Kappaphycus alvarezii extract, ceratonia siliqua fruit extract, Brassica alba sprout extract and any combinations thereof. In some embodiments of any of the compositions of the present disclosure, the cosmetically acceptable ingredient comprises Caesalpinia spinosa fruit extract.

In one aspect, the present disclosure provides a method of alleviating a cosmetic concern of a lip or a nail of an individual, the method comprises: providing a topical formulation comprising a first defensin and a cosmetically acceptable ingredient, applying the topical formulation to the lip or nail of the individual, and the topical formulation alleviates the cosmetic concern of the lip or nail of the individual.

In some embodiments, the topical formulation is applied to the lip or skin around the lip and the topical formulation alleviates the cosmetic concern of the lip. In some embodiments, the cosmetic concern of the lip comprises wrinkles on the lip, wrinkles around contour of the lip, an appearance of aging of the lip, discoloration of the lip, dryness of the lip, or any combinations thereof.

In some embodiments, the topical formulation is applied to the nail, and the topical formulation alleviates the cosmetic concern of the nail or stimulates the regeneration of the nail. In some embodiments, the nail comprises a nail portion selected from the group consisting of nail root, eponychium, proximal nail fold, lanula, cuticle, lateral nail fold, nail body/plate and hyponychium. In some embodiments, the cosmetic concern of the nail comprises brittle nail, thin nail, weakness of nail, nail discoloration, slow nail growth, nail surface irregularities, nail surface smoothness, nail vertical ridges, nail horizontal ridges, nail hydration, cuticle hydration, aesthetic appearance of the nail, aesthetic appearance of the cuticle, or any combinations thereof.

In some embodiments of any of the methods of the present disclosure, the first defensin is selected from the group consisting of alpha-defensin 1, alpha-defensin 5, alpha-defensin 6, neutrophil defensin 1, neutrophil defensin 2, neutrophil defensin 3, neutrophil defensin 4, theta-defensin, beta-defensin 1, beta-defensin 2, beta-defensin 3 and beta-defensin 4. In some embodiments of any of the methods of the present disclosure, the first defensin is present in a concentration effective to recruit Lgr6+ stem cells. In some embodiments of any of the methods of the present disclosure, the first defensin is present in a concentration of about 0.01 ng/ml to about 1000 ng/ml. In some embodiments of any of the methods of the present disclosure, the first defensin is present in a concentration of about 0.01 ng/ml to about 100 ng/ml. In some embodiments of any of the methods of the present disclosure, the first defensin is present in a concentration of about 1 ng/ml to about 100 ng/ml.

In some embodiments of any of the methods of the present disclosure, the topical formulation further comprises a second defensin, wherein the second defensin is selected from the group consisting of alpha-defensin 1, alpha-defensin 5, alpha-defensin 6, neutrophil defensin 1, neutrophil defensin 2, neutrophil defensin 3, neutrophil defensin 4, theta-defensin, beta-defensin 1, beta-defensin 2, beta-defensin 3 and beta-defensin 4. In some embodiments, the second defensin is present in a concentration effective to recruit Lgr6+ stem cells. In some embodiments, the second defensin is present in a concentration of about 0.01 ng/ml to about 1000 ng/ml. In some embodiments, the second defensin is present in a concentration of about 0.01 ng/ml to about 100 ng/ml. In some embodiments, the second defensin is present in a concentration of about 1 ng/ml to about 100 ng/ml. In some embodiments, the first defensin and the second defensin are present in a mass ratio of about 1:10 to about 10:1. In some embodiments, the first defensin and the second defensin are present in a mass ratio of about 1:5 to about 5:1.

In some embodiments of any of the methods of the present disclosure, the cosmetically acceptable ingredient is selected from the group consisting of Leuconostoc/radish root ferment filtrate, albumin, l-alanyl-l-glutamine, phospholipids, tocopheryl acetate, ubiquinone, Caesalpinia spinosa fruit extract, Helianthus annuus sprout extract, Pistacia lentiscus gum, squalane, panthenol, biotin, niacinamide, d-l methionine and any combinations thereof. In some embodiments of any of the methods of the present disclosure, the cosmetically acceptable ingredient is selected from the group consisting of Leuconostoc/radish root ferment filtrate, albumin, l-alanyl-l-glutamine, phospholipids, tocopheryl acetate, ubiquinone, Undaria pinnatifida extract, Caesalpinia spinosa fruit extract, Kappaphycus alvarezii extract, ceratonia siliqua fruit extract, Brassica alba sprout extract and any combinations thereof. In some embodiments of any of the methods of the present disclosure, the cosmetically acceptable ingredient comprises Caesalpinia spinosa fruit extract.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A depicts the surface evaluation of horizontal and vertical ridges present on nail plates of subject's nails prior to and following use of the nail composition of the present disclosure.

FIG. 1B shows a table of average reduction data for horizontal and vertical ridges on nails during a Visioscan® evaluation.

FIG. 2A depicts the surface evaluation of nail plate surface smoothness of subject's nails prior to and following use of the nail composition of the present disclosure.

FIG. 2B shows a table of average reduction data for uneven nail surface via Visioscan®.

FIG. 3A depicts evaluation of nail plate hardness of subject's nails prior to and following use of the nail composition of the present disclosure.

FIG. 3B shows a table of average increase data for nail plate hardness via Durometer Evaluation.

FIG. 4A depicts analysis of nail free edge thickness of subject's nails prior to and following use of the nail composition of the present disclosure.

FIG. 4B shows a table of average reduction data for nail free edge thickness.

FIG. 5A depicts analysis of nail free edge length of subject's nails prior to and following use of the nail composition of the present disclosure.

FIG. 5B shows a table of average increase data for nail free edge length.

FIG. 6A depicts evaluation of the hydration (e.g., electroconductivity) of the nail bed (e.g., skin around the nail) of subject's nails prior to and following use of the nail composition of the present disclosure.

FIG. 6B shows a table of average increase electroconductivity data for skin around the nail.

FIG. 7 shows a table of lip fullness characteristics and a corresponding lip fullness grading scale.

### DETAILED DESCRIPTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the invention can be described in the context of a single embodiment, the features can also be provided separately or in any suitable combination. Conversely, although the invention can be described herein in the context of separate embodiments for clarity, the invention can also be implemented in a single embodiment.

### Definitions

As used herein, the term “ready-to-use” indicates that the defensin-containing topical formulation is in a form that is presented for sale and application. It is contemplated that ready-to-use formulations can comprise a fully combined solution, cream, gel, serum, lotion, etc. Alternatively, the defensin can be packaged in a separate container (e.g., in a vial that pumps a defensin solution with a cream that the user blends before applying to unbroken skin) and combined with another topical formulation at the time of use/application.

As used herein, the phrase “sub-antimicrobially effective concentration” means concentration(s) of defensins which are characterized by an inability to inhibit the proliferation of microbes in an established infection.

As used herein, when the term “about” is used in conjunction with a numeral, “about” means a range of plus or minus ten percent of the numerical value given, including end points. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “non-injured” skin refers to skin in which dermis and hypodermis are substantially intact. Therefore, viewed from a different perspective, non-injured skin will appear intact to the unaided eye, with no breach sufficiently large or deep to result in bleeding. Thus, non-injured (or “healthy”) skin includes aged skin and skin with first degree sunburn, environmental exposure, bruising, or partially ablated stratum corneum. Non-injured (or healthy) skin also excludes skin displaying persistent infection with pathogens that result in visible symptoms and signs of infection.

As used herein, the term “Caesalpinia spinosa” or “tara” refers to a plant species native to Peru. Extracts of the tara plant can be made from the leaves, the fruit, the roots, or any combination thereof. Extracts from the tara plant can induce the formation of a protective form on the surface of human skin, reduce moisture loss, and skin peeling.

As used herein, the term “Leuconostoc/Radish Root Ferment Filtrate” refers to a chemical which is derived from the fermented root of the radish plant, Raphanus sativus. The root of the radish plant is fermented with the microorganism Leuconostoc. Leuconostoc/Radish Root Ferment Filtrate can enhance the stability and longevity of formulation. In addition, Leuconostoc/Radish Root Ferment Filtrate can have moisturizing properties.

As used herein, the term “Helianthus annuus sprout extract” refers to extract made from the seeds of a sunflower. Sunflower seed extracts can have antioxidant, antimicrobial, antidiabetic, antihypertensive, anti-inflammatory, and wound-healing properties.

As used herein, the term “Pistacia lentiscus gum” refers to the bark of the Pistacia lentiscus tree. The Pistacia lentiscus gum can block the activity of 5a reductase type I, which has an important role in sebum production.

As used herein, the term “Undaria pinnatifida extract” refers to extract made from the Wakame/sea mustard brown algae. Wakame extracts can prevent water loss and promote hydration.

As used herein, the term “Kappaphycus alvarezii extract” refers to extract made from the K. alvarezii macroalgae. K. alvarezii extract can help regulate skin barrier homeostasis.

As used herein, the term “Ceratonia siliqua fruit extract” refers to extract made from the fruit of the C. siliqua tree. The C. siliqua tree is also known as a carob tree, and the fruit of this tree is referred to as “Locust bean” or “carob tree fruit.” The Carob tree fruit extract can be used to thicken and stabilize formulations.

As used herein, the term “Brassica alba sprout extract” refers to extract made from yellow mustard. The extract can be made with the yellow mustard seeds. The extract can be made with the yellow mustard leaves. The yellow mustard extract can increase hydration.

### Topical Formulations

One aspect of the present disclosure provides a composition comprising a topical formulation. In some embodiments, the topical formulation can have a first defensin and a cosmetically acceptable ingredient. In some embodiments, the topical formulation can be formulated for use on a lip or nail. In some cases, the topical formulation can be formulated for use on a lip. In some examples, the topical formulation is applied to the surface of the lip. In some cases, the topical formulation can be formulated for use on a nail. In some examples, the topical formulation is applied to a surface of the nail, an end of the nail, a nail fold of the nail, and/or a nail root of the nail. In some embodiments, a composition can comprise at least one defensin present at a sub-antimicrobially effective concentration.

In some embodiments, the topical formulation can be formulated for use on a subject. In some cases, the subject is a mammal. In some cases, the mammal can be a non-human primate, a human, a dog, a cat, a rabbit, a rodent, or any combination thereof. In some cases, the subject is a human.

In some embodiments, the topical formulation can be applied yearly. In some embodiments, the topical formulation can be applied every six months. In some embodiments, the topical formulation can be applied every four months. In some embodiments, the topical formulation can be applied every three months. In some embodiments, the topical formulation can be applied every two months. In some embodiments, the topical formulation can be applied monthly. In some embodiments, the topical formulation can be applied biweekly. In some embodiments, the topical formulation can be applied weekly. In some embodiments, the topical formulation can be applied daily.

In some embodiments, the topical formulation can be applied between about one time to about 50 times per day. In some cases, the topical formulation can be applied at least about one time per day, at least about two times per day, at least about three times per day, at least about four times per day, at least about five times per day, at least about six times per day, at least about seven times per day, at least about eight times per day, at least about nine times per day, at least about 10 times per day, at least about 11 times per day, at least about 12 times per day, at least about 13 times per day, at least about 14 times per day, at least about 15 times per day, at least about 16 times per day, at least about 17 times per day, at least about 18 times per day, at least about 19 times per day, at least about 20 times per day, at least about 21 times per day, at least about 22 times per day, at least about 23 times per day, at least about 24 times per day, at least about 25 times per day, at least about 26 times per day, at least about 27 times per day, at least about 28 times per day, at least about 29 times per day, at least about 30 times per day, at least about 31 times per day, at least about 32 times per day, at least about 33 times per day, at least about 34 times per day, at least about 35 times per day, at least about 36 times per day, at least about 37 times per day, at least about 38 times per day, at least about 39 times per day, at least about 40 times per day, at least about 41 times per day, at least about 42 times per day, at least about 43 times per day, at least about 44 times per day, at least about 45 times per day, at least about 46 times per day, at least about 47 times per day, at least about 48 times per day, at least about 49 times per day, at least about 50 times per day, or more. In some cases, the topical formulation can be applied at most about 50 times per day, at most about 49 times per day, at most about 48 times per day, at most about 47 times per day, at most about 46 times per day, at most about 45 times per day, at most about 44 times per day, at most about 43 times per day, at most about 42 times per day, at most about 41 times per day, at most about 40 times per day, at most about 39 times per day, at most about 38 times per day, at most about 37 times per day, at most about 36 times per day, at most about 35 times per day, at most about 34 times per day, at most about 33 times per day, at most about 32 times per day, at most about 31 times per day, at most about 30 times per day, at most about 29 times per day, at most about 28 times per day, at most about 27 times per day, at most about 26 times per day, at most about 25 times per day, at most about 24 times per day, at most about 23 times per day, at most about 22 times per day, at most about 21 times per day, at most about 20 times per day, at most about 19 times per day, at most about 18 times per day, at most about 17 times per day, at most about 16 times per day, at most about 15 times per day, at most about 14 times per day, at most about 13 times per day, at most about 12 times per day, at most about 11 times per day, at most about 10 times per day, at most about 9 times per day, at most about 8 times per day, at most about 7 times per day, at most about 6 times per day, at most about 5 times per day, at most about 4 times per day, at most about 3 times per day, at most about 2 times per day, at most about 1 time per day, or less. In some cases, the topical formulation can be applied about one time per day, about two times per day, about three times per day, about four times per day, about five times per day, about six times per day, about seven times per day, about eight times per day, about nine times per day, about ten times per day, about 11 times per day, about 12 times per day, about 13 times per day, about 14 times per day, about 15 times per day, about 16 times per day, about 17 times per day, about 18 times per day, about 19 times per day, about 20 times per day, about 21 times per day, about 22 times per day, about 23 times per day, about 24 times per day, about 25 times per day, about 26 times per day, about 27 times per day, about 28 times per day, about 29 times per day, about 30 times per day, about 31 times per day, about 32 times per day, about 33 times per day, about 34 times per day, about 35 times per day, about 36 times per day, about 37 times per day, about 38 times per day, about 39 times per day, about 40 times per day, about 41 times per day, about 42 times per day, about 43 times per day, about 44 times per day, about 45 times per day, about 46 times per day, about 47 times per day, about 48 times per day, about 49 times per day, or about 50 times per day.

In some embodiments, the topical formulation can be applied between about one time to about 100 times per week. In some cases, the topical formulation is applied at least about one time per week, at least about two times per week, at least about three times per week, at least about four times per week, at least about five times per week, at least about six times per week, at least about seven times per week, at least about eight times per week, at least about nine times per week, at least about ten times per week, at least about 11 times per week, at least about 12 times per week, at least about 13 times per week, at least about 14 times per week, at least about 15 times per week, at least about 16 times per week, at least about 17 times per week, at least about 18 times per week, at least about 19 times per week, at least about 20 times per week, at least about 21 times per week, at least about 22 times per week, at least about 23 times per week, at least about 24 times per week, at least about 25 times per week, at least about 26 times per week, at least about 27 times per week, at least about 28 times per week, at least about 29 times per week, at least about 30 times per week, at least about 31 times per week, at least about 32 times per week, at least about 33 times per week, at least about 34 times per week, at least about 35 times per week, at least about 36 times per week, at least about 37 times per week, at least about 38 times per week, at least about 39 times per week, at least about 40 times per week, at least about 41 times per week, at least about 42 times per week, at least about 43 times per week, at least about 44 times per week, at least about 45 times per week, at least about 46 times per week, at least about 47 times per week, at least about 48 times per week, at least about 49 times per week, at least about 50 times per week, at least about 51 times per week, at least about 52 times per week, at least about 53 times per week, at least about 54 times per week, at least about 55 times per week, at least about 56 times per week, at least about 57 times per week, at least about 58 times per week, at least about 59 times per week, at least about 60 times per week, at least about 61 times per week, at least about 62 times per week, at least about 63 times per week, at least about 64 times per week, at least about 65 times per week, at least about 66 times per week, at least about 67 times per week, at least about 68 times per week, at least about 69 times per week, at least about 70 times per week, at least about 71 times per week, at least about 72 times per week, at least about 73 times per week, at least about 74 times per week, at least about 75 times per week, at least about 76 times per week, at least about 77 times per week, at least about 78 times per week, at least about 79 times per week, at least about 80 times per week, at least about 81 times per week, at least about 82 times per week, at least about 83 times per week, at least about 84 times per week, at least about 85 times per week, at least about 86 times per week, at least about 87 times per week, at least about 88 times per week, at least about 89 times per week, at least about 90 times per week, at least about 91 times per week, at least about 92 times per week, at least about 93 times per week, at least about 94 times per week, at least about 95 times per week, at least about 96 times per week, at least about 97 times per week, at least about 98 times per week, at least about 99 times per week, at least about 100 times per week, or more. In some cases, the topical formulation is applied at most about 100 times per week, at most about 99 times per week, at most about 98 times per week, at most about 97 times per week, at most about 96 times per week, at most about 95 times per week, at most about 94 times per week, at most about 93 times per week, at most about 92 times per week, at most about 91 times per week, at most about 90 times per week, at most about 89 times per week, at most about 88 times per week, at most about 87 times per week, at most about 86 times per week, at most about 85 times per week, at most about 84 times per week, at most about 83 times per week, at most about 82 times per week, at most about 81 times per week, at most about 80 times per week, at most about 79 times per week, at most about 78 times per week, at most about 77 times per week, at most about 76 times per week, at most about 75 times per week, at most about 74 times per week, at most about 73 times per week, at most about 72 times per week, at most about 71 times per week, at most about 70 times per week, at most about 69 times per week, at most about 68 times per week, at most about 67 times per week, at most about 66 times per week, at most about 65 times per week, at most about 64 times per week, at most about 63 times per week, at most about 62 times per week, at most about 61 times per week, at most about 60 times per week, at most about 59 times per week, at most about 58 times per week, at most about 57 times per week, at most about 56 times per week, at most about 55 times per week, at most about 54 times per week, at most about 53 times per week, at most about 52 times per week, at most about 51 times per week, at most about 50 times per week, at most about 49 times per week, at most about 48 times per week, at most about 47 times per week, at most about 46 times per week, at most about 45 times per week, at most about 44 times per week, at most about 43 times per week, at most about 42 times per week, at most about 41 times per week, at most about 40 times per week, at most about 39 times per week, at most about 38 times per week, at most about 37 times per week, at most about 36 times per week, at most about 35 times per week, at most about 34 times per week, at most about 33 times per week, at most about 32 times per week, at most about 31 times per week, at most about 30 times per week, at most about 29 times per week, at most about 28 times per week, at most about 27 times per week, at most about 26 times per week, at most about 25 times per week, at most about 24 times per week, at most about 23 times per week, at most about 22 times per week, at most about 21 times per week, at most about 20 times per week, at most about 19 times per week, at most about 18 times per week, at most about 17 times per week, at most about 16 times per week, at most about 15 times per week, at most about 14 times per week, at most about 13 times per week, at most about 12 times per week, at most about 11 times per week, at most about 10 times per week, at most about 9 times per week, at most about 8 times per week, at most about 7 times per week, at most about 6 times per week, at most about 5 times per week, at most about 4 times per week, at most about 3 times per week, at most about 2 times per week, at most about 1 time per week, or less. In some cases, the topical formulation is applied about one time per week, about two times per week, about three times per week, about four times per week, about five times per week, about six times per week, about seven times per week, about eight times per week, about nine times per week, about ten times per week, about 11 times per week, about 12 times per week, about 13 times per week, about 14 times per week, about 15 times per week, about 16 times per week, about 17 times per week, about 18 times per week, about 19 times per week, about 20 times per week, about 21 times per week, about 22 times per week, about 23 times per week, about 24 times per week, about 25 times per week, about 26 times per week, about 27 times per week, about 28 times per week, about 29 times per week, about 30 times per week, about 31 times per week, about 32 times per week, about 33 times per week, about 34 times per week, about 35 times per week, about 36 times per week, about 37 times per week, about 38 times per week, about 39 times per week, about 40 times per week, about 41 times per week, about 42 times per week, about 43 times per week, about 44 times per week, about 45 times per week, about 46 times per week, about 47 times per week, about 48 times per week, about 49 times per week, about 50 times per week, about 51 times per week, about 52 times per week, about 53 times per week, about 54 times per week, about 55 times per week, about 56 times per week, about 57 times per week, about 58 times per week, about 59 times per week, about 60 times per week, about 61 times per week, about 62 times per week, about 63 times per week, about 64 times per week, about 65 times per week, about 66 times per week, about 67 times per week, about 68 times per week, about 69 times per week, about 70 times per week, about 71 times per week, about 72 times per week, about 73 times per week, about 74 times per week, about 75 times per week, about 76 times per week, about 77 times per week, about 78 times per week, about 79 times per week, about 80 times per week, about 81 times per week, about 82 times per week, about 83 times per week, about 84 times per week, about 85 times per week, about 86 times per week, about 87 times per week, about 88 times per week, about 89 times per week, about 90 times per week, about 91 times per week, about 92 times per week, about 93 times per week, about 94 times per week, about 95 times per week, about 96 times per week, about 97 times per week, about 98 times per week, about 99 times per week, or about 100 times per week.

In some embodiments, the topical formulation can be applied between about one time to about 1,00 times per month. In some cases, the topical formulation can be applied at least about one time per month, at least about two times per month, at least about three times per month, at least about four times per month, at least about five times per month, at least about six times per month, at least about seven times per month, at least about eight times per month, at least about nine times per month, at least about ten times per month, at least about 11 times per month, at least about 12 times per month, at least about 13 times per month, at least about 14 times per month, at least about 15 times per month, at least about 16 times per month, at least about 17 times per month, at least about 18 times per month, at least about 19 times per month, at least about 20 times per month, at least about 21 times per month, at least about 22 times per month, at least about 23 times per month, at least about 24 times per month, at least about 25 times per month, at least about 26 times per month, at least about 27 times per month, at least about 28 times per month, at least about 29 times per month, at least about 30 times per month, at least about 31 times per month, at least about 32 times per month, at least about 33 times per month, at least about 34 times per month, at least about 35 times per month, at least about 36 times per month, at least about 37 times per month, at least about 38 times per month, at least about 39 times per month, at least about 40 times per month, at least about 41 times per month, at least about 42 times per month, at least about 43 times per month, at least about 44 times per month, at least about 45 times per month, at least about 46 times per month, at least about 47 times per month, at least about 48 times per month, at least about 49 times per month, at least about 50 times per month, at least about 51 times per month, at least about 52 times per month, at least about 53 times per month, at least about 54 times per month, at least about 55 times per month, at least about 56 times per month, at least about 57 times per month, at least about 58 times per month, at least about 59 times per month, at least about 60 times per month, at least about 61 times per month, at least about 62 times per month, at least about 63 times per month, at least about 64 times per month, at least about 65 times per month, at least about 66 times per month, at least about 67 times per month, at least about 68 times per month, at least about 69 times per month, at least about 70 times per month, at least about 71 times per month, at least about 72 times per month, at least about 73 times per month, at least about 74 times per month, at least about 75 times per month, at least about 76 times per month, at least about 77 times per month, at least about 78 times per month, at least about 79 times per month, at least about 80 times per month, at least about 81 times per month, at least about 82 times per month, at least about 83 times per month, at least about 84 times per month, at least about 85 times per month, at least about 86 times per month, at least about 87 times per month, at least about 88 times per month, at least about 89 times per month, at least about 90 times per month, at least about 91 times per month, at least about 92 times per month, at least about 93 times per month, at least about 94 times per month, at least about 95 times per month, at least about 96 times per month, at least about 97 times per month, at least about 98 times per month, at least about 99 times per month, at least about 100 times per month, or more. In some cases, the topical formulation can be applied at most about 100 times per month, at most about 99 times per month, at most about 98 times per month, at most about 97 times per month, at most about 96 times per month, at most about 95 times per month, at most about 94 times per month, at most about 93 times per month, at most about 92 times per month, at most about 91 times per month, at most about 90 times per month, at most about 89 times per month, at most about 88 times per month, at most about 87 times per month, at most about 86 times per month, at most about 85 times per month, at most about 84 times per month, at most about 83 times per month, at most about 82 times per month, at most about 81 times per month, at most about 80 times per month, at most about 79 times per month, at most about 78 times per month, at most about 77 times per month, at most about 76 times per month, at most about 75 times per month, at most about 74 times per month, at most about 73 times per month, at most about 72 times per month, at most about 71 times per month, at most about 70 times per month, at most about 69 times per month, at most about 68 times per month, at most about 67 times per month, at most about 66 times per month, at most about 65 times per month, at most about 64 times per month, at most about 63 times per month, at most about 62 times per month, at most about 61 times per month, at most about 60 times per month, at most about 59 times per month, at most about 58 times per month, at most about 57 times per month, at most about 56 times per month, at most about 55 times per month, at most about 54 times per month, at most about 53 times per month, at most about 52 times per month, at most about 51 times per month, at most about 50 times per month, at most about 49 times per month, at most about 48 times per month, at most about 47 times per month, at most about 46 times per month, at most about 45 times per month, at most about 44 times per month, at most about 43 times per month, at most about 42 times per month, at most about 41 times per month, at most about 40 times per month, at most about 39 times per month, at most about 38 times per month, at most about 37 times per month, at most about 36 times per month, at most about 35 times per month, at most about 34 times per month, at most about 33 times per month, at most about 32 times per month, at most about 31 times per month, at most about 30 times per month, at most about 29 times per month, at most about 28 times per month, at most about 27 times per month, at most about 26 times per month, at most about 25 times per month, at most about 24 times per month, at most about 23 times per month, at most about 22 times per month, at most about 21 times per month, at most about 20 times per month, at most about 19 times per month, at most about 18 times per month, at most about 17 times per month, at most about 16 times per month, at most about 15 times per month, at most about 14 times per month, at most about 13 times per month, at most about 12 times per month, at most about 11 times per month, at most about 10 times per month, at most about 9 times per month, at most about 8 times per month, at most about 7 times per month, at most about 6 times per month, at most about 5 times per month, at most about 4 times per month, at most about 3 times per month, at most about 2 times per month, at most about 1 time per month, or less. In some cases, the topical formulation can be applied about one time per month, about two times per month, about three times per month, about four times per month, about five times per month, about six times per month, about seven times per month, about eight times per month, about nine times per month, about 10 times per month, about 11 times per month, about 12 times per month, about 13 times per month, about 14 times per month, about 15 times per month, about 16 times per month, about 17 times per month, about 18 times per month, about 19 times per month, about 20 times per month, about 21 times per month, about 22 times per month, about 23 times per month, about 24 times per month, about 25 times per month, about 26 times per month, about 27 times per month, about 28 times per month, about 29 times per month, about 30 times per month, about 31 times per month, about 32 times per month, about 33 times per month, about 34 times per month, about 35 times per month, about 36 times per month, about 37 times per month, about 38 times per month, about 39 times per month, about 40 times per month, about 41 times per month, about 42 times per month, about 43 times per month, about 44 times per month, about 45 times per month, about 46 times per month, about 47 times per month, about 48 times per month, about 49 times per month, about 50 times per month, about 51 times per month, about 52 times per month, about 53 times per month, about 54 times per month, about 55 times per month, about 56 times per month, about 57 times per month, about 58 times per month, about 59 times per month, about 60 times per month, about 61 times per month, about 62 times per month, about 63 times per month, about 64 times per month, about 65 times per month, about 66 times per month, about 67 times per month, about 68 times per month, about 69 times per month, about 70 times per month, about 71 times per month, about 72 times per month, about 73 times per month, about 74 times per month, about 75 times per month, about 76 times per month, about 77 times per month, about 78 times per month, about 79 times per month, about 80 times per month, about 81 times per month, about 82 times per month, about 83 times per month, about 84 times per month, about 85 times per month, about 86 times per month, about 87 times per month, about 88 times per month, about 89 times per month, about 90 times per month, about 91 times per month, about 92 times per month, about 93 times per month, about 94 times per month, about 95 times per month, about 96 times per month, about 97 times per month, about 98 times per month, about 99 times per month, or about 100 times per month.

In some embodiments, the topical formulation may have one defensin, a combination of two defensins, or a combination of three or more defensins. The defensins used may be of the same or different types and subtypes. Non-limiting examples of defensins include one or more of alpha-defensin 1, alpha-defensin 5, alpha-defensin 6, neutrophil defensin 1, neutrophil defensin 2, neutrophil defensin 3, neutrophil defensin 4, theta-defensin, beta-defensin 1, beta-defensin 2, beta-defensin 3, and beta-defensin 4. Especially preferred topical formulations contain alpha-defensin 5 and beta-defensin 3. When two more defensins are used in combination, each defensin may be present in equal quantities by mass or at mass ratios specified to achieve a desired result, such as 1:1.5, 1:2, 1:4, 1:5, etc. In some embodiments, the total concentrations of defensins used in the compositions are ineffective at inhibiting substantial proliferation of microbes in established skin infections in a therapeutically effective manner.

In some embodiments, the defensins can be from natural sources. In some embodiments, the defensins can be from synthetic sources. In some embodiments, defensins can be from both natural and synthetic sources. In some cases, defensins may be obtained from plants (e.g., Arabidopsis, pea, tobacco, and spruce), mammals, or other animals. In some cases, defensins derived from natural sources may include human defensins, monkey defensins, mouse defensins, rat defensins, bovine defensins, sheep defensins, horse defensins, rabbit defensins, swine defensins, dog defensins, and/or cat defensins.

Synthetic defensins can include defensins produced by chemical synthesis (e.g., solid phase synthesis) or by recombinant technologies (e.g., produced by recombinant bacteria, yeast, tissue cultures, plants, or animals). In some embodiments, defensin analogues such as hapivirins and diprovirins may be used. In some embodiments, the defensins can be modified to increase their activity and specificity for cosmetic improvements to the appearance of lips and nails. In some cases, defensins may be unfolded and refolded under controlled conditions to ascertain proper disulfide bond formation (which can be monitored by MS analysis and/or CD spectroscopy).

Alternatively, in some embodiments, chemical modifications (e.g., using non-natural amino acids to increase half-life time, or derivatized proteinogenic amino acids to increase lipophilicity) can be used tailor the defensins to a particular need.

In some embodiments, synthetic defensins can use orthogonal protecting groups to protect selected cysteine residues, which can then be individually deprotected and bonded with the matching target cysteine residue, leading to coordinated non-random disulfide bond formation. Use of such protecting groups in the synthetic strategy can give rise to defensins with a specific activity that is comparable to the specific activity of the native defensin. Any suitable characterization and quality control measures may be employed. Typically, the specific activity of defensins incorporated into the inventive topical formulations can be measured by purity as determined by HPLC. In some embodiments, the defensin can be between 80% and 100% pure, the defensin can be at least 90% pure, the defensin can be at least 95% pure, the defensin can be at least 99% pure, or the defensin can be at least 99.9% pure. In some embodiments, proper amino acid sequence and disulfide bond formation can be confirmed by tandem MS/MS.

In some embodiments, the first defensin can be alpha-defensin 1. In some embodiments, the first defensin can be alpha-defensin 5. In some embodiments, the first defensin can be alpha-defensin 6. In some embodiments, the first defensin can be neutrophil defensin 1. In some embodiments, the first defensin can be neutrophil defensin 2. In some embodiments, the first defensin can be neutrophil defensin 3. In some embodiments, the first defensin can be neutrophil defensin 4. In some embodiments, the first defensin can be theta-defensin. In some embodiments, the first defensin can be beta-defensin 1. In some embodiments, the first defensin can be beta-defensin 2. In some embodiments, the first defensin can be beta-defensin 3. In some embodiments, the first defensin can be beta-defensin 4.

In some embodiments, the first defensin can be present in the composition at a concentration effective to recruit LGR6+ stem cells. LGR6+ cells are epidermal stem cells that can be located in the nail matrix, and which can be involved in the production of nails and the processes of nail regeneration. In some embodiments, the first defensin can be present in the composition at a concentration of about 0.01 ng/ml to about 1000 ng/ml. In some embodiments, the first defensin can be present in the composition at a concentration of about 0.01 ng/ml to about 1000 ng/ml, about 0.01 ng/ml to about 1 ng/ml, about 1 ng/ml to about 10 ng/ml, about 10 ng/ml to about 20 ng/ml, about 20 ng/ml to about 30 ng/ml, about 30 ng/ml to about 40 ng/ml, about 40 ng/ml to about 50 ng/ml, about 50 ng/ml to about 60 ng/ml, about 60 ng/ml to about 70 ng/ml, about 70 ng/ml to about 80 ng/ml, about 80 ng/ml to about 90 ng/ml, about 90 ng/ml to about 100 ng/ml, about 100 ng/ml to about 200 ng/ml, about 200 ng/ml to about 300 ng/ml, about 300 ng/ml to about 400 ng/ml, about 400 ng/ml to about 500 ng/ml, about 500 ng/ml to about 600 ng/ml, about 600 ng/ml to about 700 ng/ml, about 700 ng/ml to about 800 ng/ml, about 800 ng/ml to about 900 ng/ml, about 900 ng/ml to about 1000 ng/ml. In some embodiments, the first defensin can be present in the composition at a concentration of about 0.01 ng/ml to about 100 ng/ml. In some embodiments, the first defensin can be present in the composition at a concentration of at least about 0.01 ng/ml, at least about 0.025 ng/ml, at least about 0.05 ng/ml, at least about 0.075 ng/ml, at least about 0.1 ng/ml, at least about 0.25 ng/ml, at least about 0.5 ng/ml, at least about 0.75 ng/ml, at least about 1 ng/ml, at least about 2 ng/ml, at least about 3 ng/ml, at least about 4 ng/ml, at least about 5 ng/ml, at least about 6 ng/ml, at least about 7 ng/ml, at least about 8 ng/ml, at least about 9 ng/ml, at least about 10 ng/ml, at least about 15 ng/ml, at least about 20 ng/ml, at least about 25 ng/ml, at least about 30 ng/ml, at least about 35 ng/ml, at least about 40 ng/ml, at least about 45 ng/ml, at least about 50 ng/ml, at least about 55 ng/ml, at least about 60 ng/ml, at least about 65 ng/ml, at least about 70 ng/ml, at least about 75 ng/ml, at least about 80 ng/ml, at least about 85 ng/ml, at least about 90 ng/ml, at least about 95 ng/ml, at least about 100 ng/ml, at least about 110 ng/ml, at least about 120 ng/ml, at least about 130 ng/ml, at least about 140 ng/ml, at least about 150 ng/ml, at least about 160 ng/ml, at least about 170 ng/ml, at least about 180 ng/ml, at least about 190 ng/ml, at least about 200 ng/ml, at least about 210 ng/ml, at least about 220 ng/ml, at least about 230 ng/ml, at least about 240 ng/ml, at least about 250 ng/ml, at least about 260 ng/ml, at least about 270 ng/ml, at least about 280 ng/ml, at least about 290 ng/ml, at least about 300 ng/ml, at least about 310 ng/ml, at least about 320 ng/ml, at least about 330 ng/ml, at least about 340 ng/ml, at least about 350 ng/ml, at least about 360 ng/ml, at least about 370 ng/ml, at least about 380 ng/ml, at least about 390 ng/ml, at most about 400 ng/ml, at least about 410 ng/ml, at least about 420 ng/ml, at least about 420 ng/ml, at least about 430 ng/ml, at least about 440 ng/ml, at least about 450 ng/ml, at least about 460 ng/ml, at least about 470 ng/ml, at least about 480 ng/ml, at least about 490 ng/ml, at least about 500 ng/ml, at least about 510 ng/ml, at least about 520 ng/ml, at least about 520 ng/ml, at least about 520 ng/ml, at least about 530 ng/ml, at least about 540 ng/ml, at least about 550 ng/ml, at least about 560 ng/ml, at least about 570 ng/ml, at least about 580 ng/ml, at least about 590 ng/ml, at least about 600 ng/ml, at least about 610 ng/ml, at least about 620 ng/ml, at least about 630 ng/ml, at least about 640 ng/ml, at least about 650 ng/ml, at least about 660 ng/ml, at least about 670 ng/ml, at least about 680 ng/ml, at least about 690 ng/ml, at least about 700 ng/ml, at least about 710 ng/ml, at least about 720 ng/ml, at least about 730 ng/ml, at least about 740 ng/ml, at least about 750 ng/ml, at least about 760 ng/ml, at least about 770 ng/ml, at least about 780 ng/ml, at least about 790 ng/ml, at least about 800 ng/ml, at least about 810 ng/ml, at least about 820 ng/ml, at least about 830 ng/ml, at least about 840 ng/ml, at least about 850 ng/ml, at least about 860 ng/ml, at least about 870 ng/ml, at least about 880 ng/ml, at least about 890 ng/ml, at least about 900 ng/ml, at least about 910 ng/ml, at least about 920 ng/ml, at least about 930 ng/ml, at least about 940 ng/ml, at least about 950 ng/ml, at least about 960 ng/ml, at least about 970 ng/ml, at least about 980 ng/ml, at least about 990 ng/ml, at least about 1000 ng/ml, or more.

In some embodiments, the first defensin can be present in the composition at a concentration of at most about 1000 ng/ml, at most about 990 ng/ml, at most about 980 ng/ml, at most about 970 ng/ml, at most about 960 ng/ml, at most about 950 ng/ml, at most about 940 ng/ml, at most about 930 ng/ml, at most about 920 ng/ml, at most about 910 ng/ml, at most about 900 ng/ml, at most about 890 ng/ml, at most about 880 ng/ml, at most about 870 ng/ml, at most about 860 ng/ml, at most about 850 ng/ml, at most about 840 ng/ml, at most about 830 ng/ml, at most about 820 ng/ml, at most about 810 ng/ml, at most about 800 ng/ml, at most about 790 ng/ml, at most about 780 ng/ml, at most about 770 ng/ml, at most about 760 ng/ml, at most about 750 ng/ml, at most about 740 ng/ml, at most about 730 ng/ml, at most about 720 ng/ml, at most about 710 ng/ml, at most about 700 ng/ml, at most about 690 ng/ml, at most about 680 ng/ml, at most about 670 ng/ml, at most about 660 ng/ml, at most about 650 ng/ml, at most about 640 ng/ml, at most about 630 ng/ml, at most about 620 ng/ml, at most about 610 ng/ml, at most about 600 ng/ml, at most about 590 ng/ml, at most about 580 ng/ml, at most about 570 ng/ml, at most about 560 ng/ml, at most about 550 ng/ml, at most about 540 ng/ml, at most about 530 ng/ml, at most about 520 ng/ml, at most about 510 ng/ml, at most about 500 ng/ml, at most about 490 ng/ml, at most about 480 ng/ml, at most about 470 ng/ml, at most about 460 ng/ml, at most about 450 ng/ml, at most about 440 ng/ml, at most about 430 ng/ml, at most about 420 ng/ml, at most about 410 ng/ml, at most about 400 ng/ml, at most about 390 ng/ml, at most about 380 ng/ml, at most about 370 ng/ml, at most about 360 ng/ml, at most about 350 ng/ml, at most about 340 ng/ml, at most about 330 ng/ml, at most about 320 ng/ml, at most about 310 ng/ml, at most about 300 ng/ml, at most about 290 ng/ml, at most about 280 ng/ml, at most about 270 ng/ml, at most about 260 ng/ml, at most about 250 ng/ml, at most about 240 ng/ml, at most about 230 ng/ml, at most about 220 ng/ml, at most about 210 ng/ml, at most about 200 ng/ml, at most about 190 ng/ml, at most about 180 ng/ml, at most about 170 ng/ml, at most about 160 ng/ml, at most about 150 ng/ml, at most about 140 ng/ml, at most about 130 ng/ml, at most about 120 ng/ml, at most about 110 ng/ml, at most about 100 ng/ml, at most about 95 ng/ml, at most about 90 ng/ml, at most about 85 ng/ml, at most about 80 ng/ml, at most about 75 ng/ml, at most about 70 ng/ml, at most about 65 ng/ml, at most about 60 ng/ml, at most about 55 ng/ml, at most about 50 ng/ml, at most about 45 ng/ml, at most about 40 ng/ml, at most about 35 ng/ml, at most about 30 ng/ml, at most about 25 ng/ml, at most about 20 ng/ml, at most about 15 ng/ml, at most about 10 ng/ml, at most about 9 ng/ml, at most about 8 ng/ml, at most about 7 ng/ml, at most about 6 ng/ml, at most about 5 ng/ml, at most about 4 ng/ml, at most about 3 ng/ml, at most about 2 ng/ml, at most 1 ng/ml, at most 0.75 ng/ml, at most about 0.5 ng/ml, at most about 0.25 ng/ml, at most about 0.1 ng/ml, at most about 0.075 ng/ml, at most about 0.05 ng/ml, at most about 0.025 ng/ml, at most about 0.01 ng/ml, or less.

In some embodiments, the topical formulation can further have a second defensin. In some embodiments, the second defensin can be alpha-defensin 1. In some embodiments, the second defensin can be alpha-defensin 5. In some embodiments the second defensin can be alpha-defensin 6. In some embodiments, the second defensin neutrophil defensin 1. In some embodiments, the second defensin can be neutrophil defensin 2. In some embodiments, the second defensin can be neutrophil defensin 3. In some embodiments, the second defensin can be neutrophil defensin 4. In some embodiments, the second defensin can be theta-defensin. In some embodiments, the second defensin can be beta-defensin 1. In some embodiments, the second defensin can be beta-defensin 2. In some embodiments, the second defensin can be beta-defensin 3. In some embodiments, the second defensin can beta-defensin 4.

In some embodiments, the second defensin can be present in the composition at a concentration effective to recruit Lgr6+ stem cells. In some embodiments, the second defensin can be present in the composition at a concentration of about 0.01 ng/ml to about 1000 ng/ml. In some embodiments, the second defensin can be present in the composition at a concentration of about 0.01 ng/ml to about 1000 ng/ml, about 0.01 ng/ml to about 1 ng/ml, about 1 ng/ml to about 10 ng/ml, about 10 ng/ml to about 20 ng/ml, about 20 ng/ml to about 30 ng/ml, about 30 ng/ml to about 40 ng/ml, about 40 ng/ml to about 50 ng/ml, about 50 ng/ml to about 60 ng/ml, about 60 ng/ml to about 70 ng/ml, about 70 ng/ml to about 80 ng/ml, about 80 ng/ml to about 90 ng/ml, about 90 ng/ml to about 100 ng/ml, about 100 ng/ml to about 200 ng/ml, about 200 ng/ml to about 300 ng/ml, about 300 ng/ml to about 400 ng/ml, about 400 ng/ml to about 500 ng/ml, about 500 ng/ml to about 600 ng/ml, about 600 ng/ml to about 700 ng/ml, about 700 ng/ml to about 800 ng/ml, about 800 ng/ml to about 900 ng/ml, about 900 ng/ml to about 1000 ng/ml. In some embodiments, the second defensin can be present in the composition at a concentration of about 0.01 ng/ml to about 100 ng/ml. In some embodiments, the second defensin can be present in the composition at a concentration of at least about 0.01 ng/ml, at least about 0.025 ng/ml, at least about 0.05 ng/ml, at least about 0.075 ng/ml, at least about 0.1 ng/ml, at least about 0.25 ng/ml, at least about 0.5 ng/ml, at least about 0.75 ng/ml, at least about 1 ng/ml, at least about 2 ng/ml, at least about 3 ng/ml, at least about 4 ng/ml, at least about 5 ng/ml, at least about 6 ng/ml, at least about 7 ng/ml, at least about 8 ng/ml, at least about 9 ng/ml, at least about 10 ng/ml, at least about 15 ng/ml, at least about 20 ng/ml, at least about 25 ng/ml, at least about 30 ng/ml, at least about 35 ng/ml, at least about 40 ng/ml, at least about 45 ng/ml, at least about 50 ng/ml, at least about 55 ng/ml, at least about 60 ng/ml, at least about 65 ng/ml, at least about 70 ng/ml, at least about 75 ng/ml, at least about 80 ng/ml, at least about 85 ng/ml, at least about 90 ng/ml, at least about 95 ng/ml, at least about 100 ng/ml, at least about 110 ng/ml, at least about 120 ng/ml, at least about 130 ng/ml, at least about 140 ng/ml, at least about 150 ng/ml, at least about 160 ng/ml, at least about 170 ng/ml, at least about 180 ng/ml, at least about 190 ng/ml, at least about 200 ng/ml, at least about 210 ng/ml, at least about 220 ng/ml, at least about 230 ng/ml, at least about 240 ng/ml, at least about 250 ng/ml, at least about 260 ng/ml, at least about 270 ng/ml, at least about 280 ng/ml, at least about 290 ng/ml, at least about 300 ng/ml, at least about 310 ng/ml, at least about 320 ng/ml, at least about 330 ng/ml, at least about 340 ng/ml, at least about 350 ng/ml, at least about 360 ng/ml, at least about 370 ng/ml, at least about 380 ng/ml, at least about 390 ng/ml, at most about 400 ng/ml, at least about 410 ng/ml, at least about 420 ng/ml, at least about 420 ng/ml, at least about 430 ng/ml, at least about 440 ng/ml, at least about 450 ng/ml, at least about 460 ng/ml, at least about 470 ng/ml, at least about 480 ng/ml, at least about 490 ng/ml, at least about 500 ng/ml, at least about 510 ng/ml, at least about 520 ng/ml, at least about 520 ng/ml, at least about 520 ng/ml, at least about 530 ng/ml, at least about 540 ng/ml, at least about 550 ng/ml, at least about 560 ng/ml, at least about 570 ng/ml, at least about 580 ng/ml, at least about 590 ng/ml, at least about 600 ng/ml, at least about 610 ng/ml, at least about 620 ng/ml, at least about 630 ng/ml, at least about 640 ng/ml, at least about 650 ng/ml, at least about 660 ng/ml, at least about 670 ng/ml, at least about 680 ng/ml, at least about 690 ng/ml, at least about 700 ng/ml, at least about 710 ng/ml, at least about 720 ng/ml, at least about 730 ng/ml, at least about 740 ng/ml, at least about 750 ng/ml, at least about 760 ng/ml, at least about 770 ng/ml, at least about 780 ng/ml, at least about 790 ng/ml, at least about 800 ng/ml, at least about 810 ng/ml, at least about 820 ng/ml, at least about 830 ng/ml, at least about 840 ng/ml, at least about 850 ng/ml, at least about 860 ng/ml, at least about 870 ng/ml, at least about 880 ng/ml, at least about 890 ng/ml, at least about 900 ng/ml, at least about 910 ng/ml, at least about 920 ng/ml, at least about 930 ng/ml, at least about 940 ng/ml, at least about 950 ng/ml, at least about 960 ng/ml, at least about 970 ng/ml, at least about 980 ng/ml, at least about 990 ng/ml, at least about 1000 ng/ml, or more.

In some embodiments, the second defensin can be present in the composition at a concentration of at most about 1000 ng/ml, at most about 990 ng/ml, at most about 980 ng/ml, at most about 970 ng/ml, at most about 960 ng/ml, at most about 950 ng/ml, at most about 940 ng/ml, at most about 930 ng/ml, at most about 920 ng/ml, at most about 910 ng/ml, at most about 900 ng/ml, at most about 890 ng/ml, at most about 880 ng/ml, at most about 870 ng/ml, at most about 860 ng/ml, at most about 850 ng/ml, at most about 840 ng/ml, at most about 830 ng/ml, at most about 820 ng/ml, at most about 810 ng/ml, at most about 800 ng/ml, at most about 790 ng/ml, at most about 780 ng/ml, at most about 770 ng/ml, at most about 760 ng/ml, at most about 750 ng/ml, at most about 740 ng/ml, at most about 730 ng/ml, at most about 720 ng/ml, at most about 710 ng/ml, at most about 700 ng/ml, at most about 690 ng/ml, at most about 680 ng/ml, at most about 670 ng/ml, at most about 660 ng/ml, at most about 650 ng/ml, at most about 640 ng/ml, at most about 630 ng/ml, at most about 620 ng/ml, at most about 610 ng/ml, at most about 600 ng/ml, at most about 590 ng/ml, at most about 580 ng/ml, at most about 570 ng/ml, at most about 560 ng/ml, at most about 550 ng/ml, at most about 540 ng/ml, at most about 530 ng/ml, at most about 520 ng/ml, at most about 510 ng/ml, at most about 500 ng/ml, at most about 490 ng/ml, at most about 480 ng/ml, at most about 470 ng/ml, at most about 460 ng/ml, at most about 450 ng/ml, at most about 440 ng/ml, at most about 430 ng/ml, at most about 420 ng/ml, at most about 410 ng/ml, at most about 400 ng/ml, at most about 390 ng/ml, at most about 380 ng/ml, at most about 370 ng/ml, at most about 360 ng/ml, at most about 350 ng/ml, at most about 340 ng/ml, at most about 330 ng/ml, at most about 320 ng/ml, at most about 310 ng/ml, at most about 300 ng/ml, at most about 290 ng/ml, at most about 280 ng/ml, at most about 270 ng/ml, at most about 260 ng/ml, at most about 250 ng/ml, at most about 240 ng/ml, at most about 230 ng/ml, at most about 220 ng/ml, at most about 210 ng/ml, at most about 200 ng/ml, at most about 190 ng/ml, at most about 180 ng/ml, at most about 170 ng/ml, at most about 160 ng/ml, at most about 150 ng/ml, at most about 140 ng/ml, at most about 130 ng/ml, at most about 120 ng/ml, at most about 110 ng/ml, at most about 100 ng/ml, at most about 95 ng/ml, at most about 90 ng/ml, at most about 85 ng/ml, at most about 80 ng/ml, at most about 75 ng/ml, at most about 70 ng/ml, at most about 65 ng/ml, at most about 60 ng/ml, at most about 55 ng/ml, at most about 50 ng/ml, at most about 45 ng/ml, at most about 40 ng/ml, at most about 35 ng/ml, at most about 30 ng/ml, at most about 25 ng/ml, at most about 20 ng/ml, at most about 15 ng/ml, at most about 10 ng/ml, at most about 9 ng/ml, at most about 8 ng/ml, at most about 7 ng/ml, at most about 6 ng/ml, at most about 5 ng/ml, at most about 4 ng/ml, at most about 3 ng/ml, at most about 2 ng/ml, at most 1 ng/ml, at most 0.75 ng/ml, at most about 0.5 ng/ml, at most about 0.25 ng/ml, at most about 0.1 ng/ml, at most about 0.075 ng/ml, at most about 0.05 ng/ml, at most about 0.025 ng/ml, at most about 0.01 ng/ml, or less.

In some embodiments, the first defensin and the second defensin can be present in the composition at a mass ratio of about 1:100 to about 100:1. In some embodiments, the first defensin and the second defensin can be present in the composition at a mass ratio of about 1:100, about 1:90, about 1:80, about 1:70, about 1:60, about 1:50, about 1:40, about 1:30, 1:20, about 1:19, about 1:18, about 1:17, about 1:16, about 1:15, about 1:14, about 1:13, about 1:12, about 1:11, about 1:10, about 1:9, about 1:8, about 1:7, about 1:6, about 1:5, about 1:4, about 1:3, about 1:2, about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 30:1, about 40:1, about 50:1, about 60:1, about 70:1, about 80:1, about 90:1, 100:1. In some cases, the first defensin and the second defensin can be present in a composition at a mass ratio of about 1:10 to about 10:1. In some cases, the first defensin and the second defensin can be present in a composition at a mass ratio of about 1:5 to about 5:1.

In some embodiments, the topical formulation of the present disclosure can increase the health and/or condition of a subject's lips. An increase in health and/or condition of a subject's lips can be evidenced by smoother lips, increased lip hydration, increased skin conductance, increased skin capacitance, decreased transepidermal water loss in the lips, decreased lip dryness, decreased dryness of lip skin or skin surrounding the lips, decreased wrinkles around the lips, decreased wrinkle depth or length around the lips, improved lip color, increased lip fullness, increased lip size, or any other measure of lip health or condition.

In some embodiments, the topical formulation of the present disclosure can increase hydration of a subject's lips. In some cases, the topical formulation can increase hydration in the subject's bottom lip. In some cases, the topical formulation can increase hydration in the subject's top lip. In some cases, the topical formulation can increase cutaneous hydration of a subject's lips. In some cases, the topical formulation can increase subcutaneous hydration of a subject's lips. In some cases, the topical formulation can increase cutaneous and subcutaneous hydration of a subject's lips. In some embodiments, the hydration of the subject's lips can be measured by a skin moisture meter. Non-limiting examples of skin moisture meters include cutometer, corneometer, novameter, or any combination thereof. In some embodiments, the hydration of the subject's lips can be measured by a physical test. In some cases, the physical test can be a pinch test. The pinch test can involve pinching the subject's lips and observing how quickly the skin returns to its original position. In some cases, the physical test can be a visual assessment. The visual assessment can involve observing the subject's lips to determine if there is any rough skin or dryness preset on the subject's lips. In some embodiments, the hydration of the subject's lips can be measured by an optical method. Non-limiting examples of optical sensors include near-infrared spectrometry, nuclear magnetic resonance, optical coherence tomography, raman spectroscopy, or any combination thereof. In some embodiments, the hydration of the subject's lips can be measured by calorimetry.

In some embodiments, the topical formulation of the present disclosure can increase overall hydration in the subject's lips. In some cases, the topical formulation can increase overall hydration in the subject's bottom lip. In some cases, the topical formulation can increase overall hydration in the subject's top lip. In some embodiments, the topical formulation of the present disclosure can increase overall hydration in the subject's lips compared to a baseline overall hydration in the subject's lips from a time point when the subject was not using the topical formulation. In some embodiments, the topical formulation of the present disclosure increases the overall hydration in a subject's lips by between about 0% to about 500%. In some cases, the topical formulation of the present disclosure increases the overall hydration in the subject's lips by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure increases the overall hydration in the subject's lips by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure increases the overall hydration in the subject's lips by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation of the present disclosure can increase overall skin conductance in the subject's lips. In some cases, the topical formulation can increase overall skin conductance in the subject's bottom lip. In some cases, the topical formulation can increase overall skin conductance in the subject's top lip. In some embodiments, the topical formulation of the present disclosure can increase overall skin conductance in the subject's lips compared to a baseline overall skin conductance in the subject's lips from a time point when the subject was not using the topical formulation. In some embodiments, the topical formulation of the present disclosure increases the overall skin conductance in a subject's lips by between about 0% to about 500%. In some cases, the topical formulation of the present disclosure increases the overall skin conductance in the subject's lips by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure increases the overall skin conductance in the subject's lips by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure increases the overall skin conductance in the subject's lips by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation of the present disclosure can increase overall skin capacitance in the subject's lips. In some cases, the topical formulation can increase overall skin capacitance in the subject's bottom lip. In some cases, the topical formulation can increase overall skin capacitance in the subject's top lip. In some embodiments, the topical formulation of the present disclosure can increase overall skin capacitance in the subject's lips compared to a baseline overall skin capacitance in the subject's lips from a time point when the subject was not using the topical formulation. In some embodiments, the topical formulation of the present disclosure increases the overall skin capacitance in a subject's lips by between about 0% to about 500%. In some cases, the topical formulation of the present disclosure increases the overall skin capacitance in the subject's lips by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure increases the overall skin capacitance in the subject's lips by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure increases the overall skin capacitance in the subject's lips by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation of the present disclosure can decrease the transepidermal water loss from the subject's lips. In some cases, the topical formulation can decrease the transepidermal water loss from the subject's bottom lip. In some cases, the topical formulation can decrease the transepidermal water loss from the subject's top lip. In some cases, the transepidermal water loss from the subject's lips can be measured by a transepidermal water loss (TEWL) probe. Non-limiting examples of TEWL probes include AquaFlux™ AF200, DermaLab, Evaporimeter 2100, GPSkin Barrier Light, GPSkin Pro, H4300, H4500, MEECO, Noevir©-EVA, Evaporimeter EP1, Tewameter® TM210, Tewameter® TM300, Vapometer™ Vapometer™ SWL-2, Vapometer™ SWL-3, or any combination thereof.

In some embodiments, the topical formulation of the present disclosure can decrease the transepidermal water loss from the subject's lips compared to a baseline transepidermal water loss from the subject's lips from a time point when the subject was not using the topical formulation. In some embodiments, the topical formulation of the present disclosure decreases the transepidermal water loss from the subject's lips by between about 0% to about 500%. In some cases, the topical formulation of the present disclosure decreases the transepidermal water loss from the subject's lips by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the transepiderminal water loss from the subject's lips by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the transepidermal water loss from the subject's lips by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation of the present disclosure can decrease the transepidermal water loss from the subject's lips by between about 0 milliliters per day (mL/day) to about 1,000 mL/day. In some cases, the topical formulation decreases the transepidermal water loss from the subject's lips by at least about 0 mL/day, at least about 10 mL/day, at least about 20 mL/day, at least about 30 mL/day, at least about 40 mL/day, at least about 50 mL/day, at least about 60 mL/day, at least about 70 mL/day, at least about 80 mL/day, at least about 90 mL/day, at least about 100 mL/day, at least about 120 mL/day, at least about 140 mL/day, at least about 160 mL/day, at least about 180 mL/day, at least about 200 mL/day, at least about 220 mL/day, at least about 240 mL/day, at least about 260 mL/day, at least about 280 mL/day, at least about 300 mL/day, at least about 320 mL/day, at least about 340 mL/day, at least about 360 mL/day, at least about 380 mL/day, at least about 400 mL/day, at least about 420 mL/day, at least about 440 mL/day, at least about 460 mL/day, at least about 480 mL/day, at least about 500 mL/day, at least about 520 mL/day, at least about 540 mL/day, at least about 560 mL/day, at least about 580 mL/day, at least about 600 mL/day, at least about 620 mL/day, at least about 640 mL/day, at least about 660 mL/day, at least about 680 mL/day, at least about 700 mL/day, at least about 720 mL/day, at least about 740 mL/day, at least about 760 mL/day, at least about 780 mL/day, at least about 800 mL/day, at least about 820 mL/day, at least about 840 mL/day, at least about 860 mL/day, at least about 880 mL/day, at least about 900 mL/day, at least about 920 mL/day, at least about 940 mL/day, at least about 960 mL/day, at least about 980 mL/day, at least about 1,000 mL/day, or more. In some cases, the topical formulation decreases the transepidermal water loss from the subject's lips by at most about 1,000 mL/day, at most about 980 mL/day, at most about 960 mL/day, at most about 940 mL/day, at most about 920 mL/day, at most about 900 mL/day, at most about 880 mL/day, at most about 860 mL/day, at most about 840 mL/day, at most about 820 mL/day, at most about 800 mL/day, at most about 780 mL/day, at most about 760 mL/day, at most about 740 mL/day, at most about 720 mL/day, at most about 700 mL/day, at most about 680 mL/day, at most about 660 mL/day, at most about 640 mL/day, at most about 620 mL/day, at most about 600 mL/day, at most about 580 mL/day, at most about 560 mL/day, at most about 540 mL/day, at most about 520 mL/day, at most about 500 mL/day, at most about 480 mL/day, at most about 460 mL/day, at most about 440 mL/day, at most about 420 mL/day, at most about 400 mL/day, at most about 380 mL/day, at most about 360 mL/day, at most about 340 mL/day, at most about 320 mL/day, at most about 300 mL/day, at most about 280 mL/day, at most about 260 mL/day, at most about 240 mL/day, at most about 220 mL/day, at most about 200 mL/day, at most about 180 mL/day, at most about 160 mL/day, at most about 140 mL/day, at most about 120 mL/day, at most about 100 mL/day, at most about 90 mL/day, at most about 80 mL/day, at most about 70 mL/day, at most about 60 mL/day, at most about 50 mL/day, at most about 40 mL/day, at most about 30 mL/day, at most about 20 mL/day, at most about 10 mL/day, at most about 0 mL/day, or less. In some cases, the topical formulation decreases transepidermal water loss in the subject's lips by about 0 mL/day, about 10 mL/day, about 20 mL/day, about 30 mL/day, about 40 mL/day, about 50 mL/day, about 60 mL/day, about 70 mL/day, about 80 mL/day, about 90 mL/day, about 100 mL/day, about 120 mL/day, about 140 mL/day, about 160 mL/day, about 180 mL/day, about 200 mL/day, about 220 mL/day, about 240 mL/day, about 260 mL/day, about 280 mL/day, about 300 mL/day, about 320 mL/day, about 340 mL/day, about 360 mL/day, about 380 mL/day, about 400 mL/day, about 420 mL/day, about 440 mL/day, about 460 mL/day, about 480 mL/day, about 500 mL/day, about 520 mL/day, about 540 mL/day, about 560 mL/day, about 580 mL/day, about 600 mL/day, about 620 mL/day, about 640 mL/day, about 660 mL/day, about 680 mL/day, about 700 mL/day, about 720 mL/day, about 740 mL/day, about 760 mL/day, about 780 mL/day, about 800 mL/day, about 820 mL/day, about 840 mL/day, about 860 mL/day, about 880 mL/day, about 900 mL/day, about 920 mL/day, about 940 mL/day, about 960 mL/day, about 980 mL/day, or about 1,000 mL/day.

In some embodiments, the topical formulation of the present disclosure can decrease dryness of the subject's lips. In some cases, the topical formulation can decrease dryness of the subject's bottom lip. In some cases, the topical formulation can decrease dryness of the subject's top lip. In some cases, the decrease of dryness of the subject's lips can be measured by visual observation. In some cases, the visual observation can comprise performing an evaluation of the subject's lips in a mirror. In some cases, the visual observation can comprise performing images of the subject's lips. In some examples, the images of the subject's lips are analyzed using image analysis software. In some cases, the visual observation can be performed by the subject and/or a separate individual. In some examples, the visual observation can comprise rating the subject's lips with a dryness score. The dryness score can include the following: a 0 dryness score corresponds to no dryness or chapping evident; a 1-2 dryness score corresponds to fine scaling; a 3-4 dryness score corresponds to coarse scaling and slight cracking; a 5-6 dryness score corresponds to coarse scaling and obvious cracking; and a 7-8 dryness score corresponds to course scaling and cracking progressing to fissuring. In some cases, the decrease of dryness of the subject's lips can be measured by physical evaluation. In some examples, the physical evaluation can comprise the subject and/or a separate individual touching the subject's lips. In some examples, the physical evaluation can comprise rating the subject's lips with a dryness score. The dryness score can include the following: a 0 dryness score correspond to smooth skin on the subject's lips; a 1-2 dryness score corresponds with slight, but definite roughness; a 3-4 dryness score corresponds with moderate rough skin on the subject's lips; a 5-6 dryness score corresponds with marked rough skin on the subject's lips; and a 7-8 dryness score corresponds with very marked rough skin on the subject's lips. In some embodiments, the decrease of dryness of the subject's lips can be measured by a visual observation and a physical evaluation.

In some embodiments, the topical formulation of the present disclosure can decrease the dryness score of the subject's lips. In some embodiments, the topical formulation of the present disclosure can decrease the dryness score of the subject's lips compared to a baseline dryness score of the subject's lips from a time point when the subject was not using the topical formulation. In some embodiments, the topical formulation of the present disclosure decreases the dryness score of the subject's lips by between about 0% to about 500%. In some cases, the topical formulation of the present disclosure decreases the dryness score of the subject's lips by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the dryness score of the subject's lips by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the dryness score of the subject's lips by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation of the present disclosure can decrease overall skin dryness of the subject's lips compared to a baseline overall skin dryness of the subject's lips from a time point when the subject was not using the topical formulation. In some embodiments, the topical formulation of the present disclosure decreases the overall skin dryness of the subject's lips by between about 0% to about 500%. In some cases, the topical formulation of the present disclosure decreases the overall skin dryness of the subject's lips by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the overall skin dryness of the subject's lips by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the overall skin dryness of the subject's lips by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation of the present disclosure can decrease wrinkles around contours of a subject's lips. In some cases, the topical formulation of the present disclosure can decrease wrinkles around the contour of the subject's bottom lip. In some cases, the topical formulation of the present disclosure can decrease wrinkles around the contour of the subject's top lip. In some cases, the topical formulation of the present disclosure can decrease wrinkles around the contour of the subject's bottom lip and around the contour of the subject's top lip. In some embodiments, the decrease of wrinkles around the contours of the subject's lips can be measured by a visual observation. The visual observation can comprise performing an evaluation of the subject's lips in a mirror. In some cases, the evaluation can include the subject rating the following wrinkle characteristics from 0 (e.g., not observed) to 10 (e.g., severe): overall number of wrinkles around the contours of the lips, depth of wrinkles around the contours of the lips, length of wrinkles around the contours of the lips, or any combination thereof. In some cases, the visual observation can comprise using the Daniell system of scoring the appearance of facial wrinkles. In some cases, the visual observation of the subject's lips can be performed by the subject and/or a separate observer. In some examples, the separate observer is a different individual from the subject. In some examples, the separate observer is a clinician. In some embodiments, the decrease of wrinkles around the contours of the subject's lips can be measured by an optical method. Non-limiting examples of optical methods to measure wrinkles around the contours of the subject's lips include image-analysis software, visual illusion-based image feature enhancement system, three-dimensional speckle tracking photogrammetry, digital fringe profilometry, three-dimensional acquisition, three-dimensional projecting grid technique, or any combination thereof.

In some embodiments, the topical formulation of the present disclosure can decrease the rating of the wrinkle characteristics of the subject's lips. In some cases, the topical formulation can decrease the rating of the wrinkle characteristics of the subject's top lip. In some cases, the topical formulation can decrease the rating of the wrinkle characteristics of the subject's bottom lip. In some embodiments, the topical formulation can decrease the rating of the wrinkle characteristics of the subject's lips compared to a baseline rating of the wrinkle characteristics of the subject's lips from a time point when the subject was not using the topical formulation. In some embodiments, the topical formulation of the present disclosure decreases the rating of the wrinkle characteristics of the subject's lips by between about 0% to about 500%. In some cases, the topical formulation of the present disclosure decreases the rating of the wrinkle characteristics of the subject's lips by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the rating of the wrinkle characteristics of the subject's lips by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the rating of the wrinkle characteristics of the subject's lips by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation can decrease a depth of the wrinkles around the contours of the subject's lips. In some embodiments, the topical formulation can decrease a depth of the wrinkles around the contours of the subject's bottom lip. In some embodiments, the topical formulation can decrease a depth of the wrinkles around the contours of the subject's top lip. In some embodiments, the topical formulation can decrease a depth of the wrinkles around the contours of the subject's lips compared to a baseline depth of the wrinkles around the contours of the subject's lips from a time point when the subject was not using the topical formulation. In some embodiments, the topical formulation of the present disclosure decreases the depth of the wrinkles around the contours of the subject's lips by between about 0% to about 500%. In some cases, the topical formulation of the present disclosure decreases the depth of the wrinkles around the contours of the subject's lips by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the depth of the wrinkles around the contours of the subject's lips by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the depth of the wrinkles around the contours of the subject's lips by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation can decrease a length of the wrinkles around the contours of the subject's lips. In some embodiments, the topical formulation can decrease a length of the wrinkles around the contours of the subject's bottom lip. In some embodiments, the topical formulation can decrease a length of the wrinkles around the contours of the subject's top lip. In some embodiments, the topical formulation can decrease a length of the wrinkles around the contours of the subject's lips compared to a baseline length of the wrinkles around the contours of the subject's lips from a time point when the subject was not using the topical formulation. In some embodiments, the topical formulation of the present disclosure decreases the length of the wrinkles around the contours of the subject's lips by between about 0% to about 500%. In some cases, the topical formulation of the present disclosure decreases the length of the wrinkles around the contours of the subject's lips by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the length of the wrinkles around the contours of the subject's lips by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the length of the wrinkles around the contours of the subject's lips by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation can decrease an overall number of wrinkles around the contours of the subject's lips. In some embodiments, the topical formulation can decrease an overall number of wrinkles around the contours of the subject's bottom lip. In some embodiments, the topical formulation can decrease an overall number of wrinkles around the contours of the subject's top lip. In some embodiments, the topical formulation can decrease an overall number of wrinkles around the contours of the subject's lips compared to a baseline overall number of wrinkles around the contours of the subject' lips from a time point when the subject was not using the topical formulation. In some embodiments, the topical formulation of the present disclosure decreases the overall number of wrinkles around the contours of the subject's lips by between about 0% to about 500%. In some cases, the topical formulation of the present disclosure decreases the overall number of wrinkles around the contours of the subject's lips by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the overall number of wrinkles around the contours of the subject's lips by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the overall number of wrinkles around the contours of the subject's lips by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation can decrease a visual appearance of wrinkles around the contours of the subject's lips. In some embodiments, the topical formulation can decrease a visual appearance of wrinkles around the contours of the subject's bottom lip. In some embodiments, the topical formulation can decrease a visual appearance of wrinkles around the contours of the subject's top lip. In some embodiments, the topical formulation can decrease a visual appearance of wrinkles around the contours of the subject's lips compared to a baseline visual appearance of the wrinkles around the contours of the subject's lips from a time point when the subject was not using the topical formulation. In some embodiments, the topical formulation of the present disclosure decreases the visual appearance of wrinkles around the contours of the subject's lips by between about 0% to about 500%. In some cases, the topical formulation of the present disclosure decreases the visual appearance of wrinkles around the contours of the subject's lips by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the visual appearance of wrinkles around the contours of the subject's lips by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the visual appearance of wrinkles around the contours of the subject's lips by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation of the present disclosure can decrease wrinkles on the subject's lips. In some cases, the topical formulation of the present disclosure can decrease wrinkles on the subject's bottom lip. In some cases, the topical formulation of the present disclosure can decrease wrinkles on the subject's top lip. In some cases, the topical formulation of the present disclosure can decrease wrinkles on the subject's bottom lip and on the subject's top lip. In some embodiments, the decrease of wrinkles on the subject's lips can be measured by a visual observation. The visual observation can comprise performing an evaluation of the subject's lips in a mirror. In some cases, the self-evaluation can include rating the following wrinkle characteristics from 0 (e.g., not observed) to 10 (e.g., severe): overall number of wrinkles on the lips, depth of wrinkles on the lips, length of wrinkles on the lips, or any combination thereof. In some cases, the visual observation can comprise using the Daniell system of scoring the appearance of facial wrinkles. In some cases, the visual observation of the subject's lips can be performed by the subject and/or a separate observer. In some embodiments, the decrease of wrinkles on the subject's lips can be measured by an optical method. Non-limiting examples of optical methods to measure wrinkles on the subject's lips include image analysis software, visual illusion-based image feature enhancement system, three-dimensional speckle tracking photogrammetry, digital fringe profilometry, three-dimensional acquisition, three-dimensional projecting grid technique, or any combination thereof.

In some embodiments, the topical formulation of the present disclosure can decrease the rating of the wrinkle characteristics of the subject's lips. In some cases, the topical formulation can decrease the rating of the wrinkle characteristics of the subject's top lip. In some cases, the topical formulation can decrease the rating of the wrinkle characteristics of the subject's bottom lip. In some embodiments, the topical formulation can decrease the rating of the wrinkle characteristics of the subject's lips compared to a baseline rating of the wrinkle characteristics of the subject's lips from a time point when the subject was not using the topical formulation. In some embodiments, the topical formulation of the present disclosure decreases the rating of the wrinkle characteristics of the subject's lips by between about 0% to about 500%. In some cases, the topical formulation of the present disclosure decreases the rating of the wrinkle characteristics of the subject's lips by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the rating of the wrinkle characteristics of the subject's lips by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the rating of the wrinkle characteristics of the subject's lips by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation can decrease a depth of the wrinkles on the subject's lips. In some embodiments, the topical formulation can decrease a depth of the wrinkles on the subject's bottom lip. In some embodiments, the topical formulation can decrease a depth of the wrinkles on the subject's top lip. In some embodiments, the topical formulation can decrease a depth of the wrinkles on the subject's lips compared to a baseline depth of the wrinkles on the subject's lips from a time point when the subject was not using the topical formulation. In some embodiments, the topical formulation of the present disclosure decreases the depth of the wrinkles on the subject's lips by between about 0% to about 500%. In some cases, the topical formulation of the present disclosure decreases the depth of the wrinkles on the subject's lips by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the depth of the wrinkles on the subject's lips by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the depth of the wrinkles on the subject's lips by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation can decrease a length of the wrinkles on the subject's lips. In some embodiments, the topical formulation can decrease a length of the wrinkles on the subject's bottom lip. In some embodiments, the topical formulation can decrease a length of the wrinkles on the subject's top lip. In some embodiments, the topical formulation can decrease a length of the wrinkles on the subject's lips compared to a baseline length of the wrinkles on the subject's lips from a time point when the subject was not using the topical formulation. In some embodiments, the topical formulation of the present disclosure decreases the length of the wrinkles on the subject's lips by between about 0% to about 500%. In some cases, the topical formulation of the present disclosure decreases the length of the wrinkles on the subject's lips by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the length of the wrinkles around on the subject's lips by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the length of the wrinkles on the subject's lips by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation can decrease an overall number of wrinkles on the subject's lips. In some embodiments, the topical formulation can decrease an overall number of wrinkles on the subject's bottom lip. In some embodiments, the topical formulation can decrease an overall number of wrinkles on the subject's top lip. In some embodiments, the topical formulation can decrease an overall number of wrinkles on the subject's lips compared to a baseline overall number of wrinkles on the subject's lips from a time point when the subject was not using the topical formulation. In some embodiments, the topical formulation of the present disclosure decreases the overall number of wrinkles on the subject's lips by between about 0% to about 500%. In some cases, the topical formulation of the present disclosure decreases the overall number of wrinkles on the subject's lips by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the overall number of wrinkles on the subject's lips by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the overall number of wrinkles on the subject's lips by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation can decrease a visual appearance of wrinkles on the subject's lips. In some embodiments, the topical formulation can decrease a visual appearance of wrinkles on the subject's bottom lip. In some embodiments, the topical formulation can decrease a visual appearance of wrinkles on the subject's top lip. In some embodiments, the topical formulation can decrease a visual appearance of wrinkles on the subject's lips compared to a baseline visual appearance of the wrinkles on the subject's lips from a time point when the subject was not using the topical formulation. In some embodiments, the topical formulation of the present disclosure decreases the visual appearance of wrinkles on the subject's lips by between about 0% to about 500%. In some cases, the topical formulation of the present disclosure decreases the visual appearance of wrinkles on the subject's lips by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the visual appearance of wrinkles on the subject's lips by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the visual appearance of wrinkles on the subject's lips by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation can improve color of the subject's lips. In some embodiments, the topical formulation can improve color of the subject's bottom lip. In some embodiments, the topical formulation can improve color of the subject's top lip. In some cases, the topical formulation can result in a healthy lip color. Healthy lip colors can include pinks lips or light red lips. Non-healthy lip colors can include pale lips, purple lips, or blue lips. In some cases, improvement of the color of the subject's lips can be measured by a visual observation. The visual observation can comprise performing an evaluation of the subject's lips in a mirror and determining the current color of the subject's lips. In some cases, the visual observation can comprise taking images (e.g., pictures) of the subject's lips and determining the color of the subject's lips in the images. In some examples, the images of the subject's lips can be analyzed via an image analysis software to determine the color of the subject's lips. In some cases, the visual observation can be performed by the subject and/or a separate observer.

In some embodiments, the topical formulation of the present disclosure can increase fullness of the subject's lips. In some cases, the topical formulation of the present disclosure can increase fullness of the subject's bottom lip. In some cases, the topical formulation of the present disclosure can increase fullness of the subject's top lip. In some embodiments, the increase in the fullness of the subject's lips can be measured by visual observation. The visual observation can comprise performing an evaluation of the subject's lips in a mirror and/or taking images of the subject's lips. In some cases, the images of the subject's lips can be analyzed with an image analysis software. Non-limiting examples of image analysis software includes: PhotoGrammetrix® Image Analysis 3D Zephyr software, PhotoModeler software, Agisoft PhotoScan software, Metashape software, Pix4Dmapper software, or any combination thereof. In some cases, the visual observation of the subject's lips can be performed by the subject and/or a separate observer.

In some cases, the visual observation comprises grading the fullness of the subject's lips. The lip fullness grading scale can include grading lip size of the subject's lips, vermillion body (e.g., pink or red portion of the lips) of the subject's lips, and/or vermillion border (e.g., edge of the pink or red portion of the lips and the surrounding facial skin) of the subject's lips. In some examples, the lip fullness grading scale of the lip size of the subject's lips can include: a lip size score of −2 corresponds to very thin lips; a lip size score of −1 corresponds to thin lips; a lip size score of 0 corresponds to medium sized lips; a lip size score of 1 corresponds to full lips; and a lip size score of 2 corresponds to extremely full lips. In some examples, the lip fullness grading scale of the vermillion body can include: a vermillion body score of −1 corresponds with tight lips with almost no lines; a vermillion body score of 0 corresponds with rounded lips with natural lines; a vermillion body score of 1 corresponds with less rounded lips with fine lines; a vermillion body score of 2 corresponds with flattening lips with moderate wrinkles; and a vermillion body score of 3 corresponds with lips with flattening lips with severe wrinkles. In some examples, the lip fullness grading scale of the vermillion border can include: a vermillion border score of −1 corresponds with protruding vermillion borders and/or creating perioral shadow; a vermillion border score of 0 corresponds to distinct and intact vermillion borders with or without shadow from the mid-lower lip; a vermillion border scale of 1 corresponds to distinct vermillion borders but broken by fine lines with or without shadow from mid-lower lip; a vermillion border scale of 2 corresponds to indistinct vermillion borders broken by moderate lines with or without shadow from mid-lower lip; and a vermillion border scale of 3 corresponds with indistinct and severely lined vermillion borders without or without shadow from mid-lower lip.

In some embodiments, the topical formulation can increase an overall fullness of the subject's lips. In some embodiments, the topical formulation can increase the overall fullness of the subject's lips compared to a baseline overall fullness of the subject's lips from a time point when the subject was not using the topical formulation. In some embodiments, the topical formulation of the present disclosure increases the overall fullness of the subject's lips by between about 0% to about 500%. In some cases, the topical formulation of the present disclosure increases the overall fullness of the subject's lips by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure increases the overall fullness of the subject's lips by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure increases the overall fullness of the subject's lips by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation can increase the lip size scale score of the subject's lips. In some embodiments, the topical formulation can increase the lip size scale score of the subject's lips compared to a baseline lip size scale score of the subject's lips from a time point when the subject was not using the topical formulation. In some embodiments, the topical formulation of the present disclosure increases the lip size scale score of the subject's lips by between about 0% to about 500%. In some cases, the topical formulation of the present disclosure increases the lip size scale score of the subject's lips by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure increases the lip size scale score of the subject's lips by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure increases the lip size scale score of the subject's lips by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation can decrease the vermillion body scale score of the subject's lips. In some embodiments, the topical formulation can increase the vermillion body scale score of the subject's lips compared to a baseline vermillion body scale score of the subject's lips from a time point when the subject was not using the topical formulation. In some embodiments, the topical formulation of the present disclosure decreases the vermillion body scale score of the subject's lips by between about 0% to about 500%. In some cases, the topical formulation of the present disclosure decreases the vermillion body scale score of the subject's lips by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the vermillion body scale score of the subject's lips by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the vermillion body scale score of the subject's lips by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation can improve the vermillion border scale score of the subject's lips. In some embodiments, the topical formulation can increase the vermillion border scale score of the subject's lips compared to a baseline vermillion border scale score of the subject's lips from a time point when the subject was not using the topical formulation. In some embodiments, the topical formulation of the present disclosure increases the vermillion border scale score of the subject's lips by between about 0% to about 500%. In some cases, the topical formulation of the present disclosure increases the vermillion border scale score of the subject's lips by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure increases the vermillion border scale score of the subject's lips by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure increases the vermillion border scale score of the subject's lips by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation of the present disclosure can evince signs of improvements in lip appearance and/or health in at least about 1 days, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 31 days, at least about 32 days, at least about 33 days, at least about 34 days, at least about 35 days, at least about 36 days, at least about 37 days, at least about 38 days, at least about 39 days, at least about 40 days, at least about 41 days, at least about 42 days, at least about 43 days, at least about 44 days, at least about 45 days, at least about 46 days, at least about 47 days, at least about 48 days, at least about 49 days, at least about 50 days, at least about 51 days, at least about 52 days, at least about 53 days, at least about 54 days, at least about 55 days, at least about 56 days, at least about 57 days, at least about 58 days, at least about 59 days, at least about 60 days, at least about 61 days, at least about 62 days, at least about 63 days, at least about 64 days, at least about 65 days, at least about 66 days, at least about 67 days, at least about 68 days, at least about 69 days, at least about 70 days, at least about 71 days, at least about 72 days, at least about 73 days, at least about 74 days, at least about 75 days, at least about 76 days, at least about 77 days, at least about 78 days, at least about 79 days, at least about 80 days, at least about 81 days, at least about 82 days, at least about 83 days, at least about 84 days, at least about 85 days, at least about 86 days, at least about 87 days, at least about 88 days, at least about 89 days, at least about 90 days, at least about 91 days, at least about 92 days, at least about 93 days, at least about 94 days, at least about 95 days, at least about 96 days, at least about 97 days, at least about 98 days, at least about 99 days, or at least about 100 days. In some embodiments, the topical formulation of the present disclosure can evince signs of improvements in lip appearance and/or health in at most about 100 days, at most about 99 days, at most about 98 days, at most about 97 days, at most about 96 days, at most about 95 days, at most about 94 days, at most about 93 days, at most about 92 days, at most about 91 days, at most about 90 days, at most about 89 days, at most about 88 days, at most about 87 days, at most about 86 days, at most about 85 days, at most about 84 days, at most about 83 days, at most about 82 days, at most about 81 days, at most about 80 days, at most about 79 days, at most about 78 days, at most about 77 days, at most about 76 days, at most about 75 days, at most about 74 days, at most about 73 days, at most about 72 days, at most about 71 days, at most about 70 days, at most about 69 days, at most about 68 days, at most about 67 days, at most about 66 days, at most about 65 days, at most about 64 days, at most about 63 days, at most about 62 days, at most about 61 days, at most about 60 days, at most about 59 days, at most about 58 days, at most about 57 days, at most about 56 days, at most about 55 days, at most about 54 days, at most about 53 days, at most about 52 days, at most about 51 days, at most about 50 days, at most about 49 days, at most about 48 days, at most about 47 days, at most about 46 days, at most about 45 days, at most about 44 days, at most about 43 days, at most about 42 days, at most about 41 days, at most about 40 days, at most about 39 days, at most about 38 days, at most about 37 days, at most about 36 days, at most about 35 days, at most about 34 days, at most about 33 days, at most about 32 days, at most about 31 days, at most about 30 days, at most about 29 days, at most about 28 days, at most about 27 days, at most about 26 days, at most about 25 days, at most about 24 days, at most about 23 days, at most about 22 days, at most about 21 days, at most about 20 days, at most about 19 days, at most about 18 days, at most about 17 days, at most about 16 days, at most about 15 days, at most about 14 days, at most about 13 days, at most about 12 days, at most about 11 days, at most about 10 days, at most about 9 days, at most about 8 days, at most about 7 days, at most about 6 days, at most about 5 days, at most about 4 days, at most about 3 days, at most about 2 days, at most about 1 day. In some embodiments, the topical formulation of the present disclosure can evince signs of improvements in lip appearance and/or health in about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, about 35 days, about 36 days, about 37 days, about 38 days, about 39 days, about 40 days, about 41 days, about 42 days, about 43 days, about 44 days, about 45 days, about 46 days, about 47 days, about 48 days, about 49 days, about 50 days, about 51 days, about 52 days, about 53 days, about 54 days, about 55 days, about 56 days, about 57 days, about 58 days, about 59 days, about 60 days, about 61 days, about 62 days, about 63 days, about 64 days, about 65 days, about 66 days, about 67 days, about 68 days, about 69 days, about 70 days, about 71 days, about 72 days, about 73 days, about 74 days, about 75 days, about 76 days, about 77 days, about 78 days, about 79 days, about 80 days, about 81 days, about 82 days, about 83 days, about 84 days, about 85 days, about 86 days, about 87 days, about 88 days, about 89 days, about 90 days, about 91 days, about 92 days, about 93 days, about 94 days, about 95 days, about 96 days, about 97 days, about 98 days, about 99 days, or about 100 days.

In some embodiments, the lip health improvements of the topical formulation of the present disclosure can last for at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 3 months, at least about 4 months, at least about 5 months, at least about 6 months, at least about 7 months, at least about 8 months, at least about 9 months, or at least about 10 months. In some embodiments, the lip health improvements of the topical formulation of the present disclosure can last for at most about 10 months, at most about 9 months, at most about 8 months, at most about 7 months, at most about 6 months, at most about 5 months, at most about 4 months, at most about 3 months, at most about 12 weeks, at most about 11 weeks, at most about 10 weeks, at most about 9 weeks, at most about 8 weeks, at most about 7 weeks, at most about 6 weeks, at most about 5 weeks, at most about 4 weeks, at most about 3 weeks, at most about 2 weeks, or at most about 1 week. In some embodiments, the lip health improvements of the topical formulation of the present disclosure can last for about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 3 months, about 4 months, about 5 months, about 6 months, about 7 months, about 8 months, about 9 months, or about 10 months.

In some embodiments, the topical formulation of the present disclosure can increase the health and/or condition of a subject's nails (e.g., fingernails, toenails). An increase in health and/or condition of a subject's nails can be evidenced by increased nail smoothness (e.g., nail surface smoothness), decreased number of nail ridges (e.g., horizontal ridges, vertical ridges), improved nail surface irregularities, decreased width and/or depth of nail ridges (e.g., horizontal ridges, vertical ridges), increased nail strength, increased nail thickness, increased nail hydration, increased nail nourishment, increased keratin production, increased antioxidant production, increased nail growth, increased nail plate hardness, increased nail fold hardness, increased nail free edge thickness, increased nail free edge length, increased nail root hydration, increased nail cuticle hydration, increased nail root conductance, increased nail root capacitance, or any other measure of nail health or condition.

In some embodiments, the topical formulation of the present disclosure can increase a thickness of a subject's nail. In some cases, the thickness of the subject's nail can be measured using a caliper. In some cases, the thickness of the subject's nail can be measured using topical coherence tomography. In some cases, the thickness of the subject's nail can be measured using ultrasonography. In some cases, the thickness of the subject's nail can be measuring using high frequency echography.

In some cases, the topical formulation can increase the thickness of a subject's nail compared to a baseline thickness of the subject's nail from a time point when the subject was not using the topical formulation. In some cases, the topical formulation increases the thickness of the subject's nail by between about 0% to about 500%. In some cases, the topical formulation increases the thickness of the subject's nail by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure increases the thickness of the subject's nail by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure increases the thickness of the subject's nail by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some cases, the topical formulation can increase the thickness of a subject's nail compared to a baseline thickness of the subject's nail from a time point when the subject was not using the topical formulation by between about 0 micrometers (μm) to about 10 millimeters (mm). In some cases, the topical formulation can increase the thickness of a subject's nail by at least about 0 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 200 μm, at least about 300 μm, at least about 400 μm, at least about 500 μm, at least about 600 μm, at least about 700 μm, at least about 800 μm, at least about 900 μm, at least about 1 mm, at least about 2 mm, at least about 3 mm, at least about 4 mm, at least about 5 mm, at least about 6 mm, at least about 7 mm, at least about 8 mm, at least about 9 mm, at least about 10 mm, or more. In some cases, the topical formulation can increase the thickness of the subject's nail by at most about 10 mm, at most about 9 mm, at most about 8 mm, at most about 7 mm, at most about 6 mm, at most about 5 mm, at most about 4 mm, at most about 3 mm, at most about 2 mm, at most about 1 mm, at most about 900 μm, at most about 800 μm, at most about 700 μm, at most about 600 μm, at most about 500 μm, at most about 400 μm, at most about 300 μm, at most about 200 μm, at most about 100 μm, at most about 90 μm, at most about 80 μm, at most about 70 μm, at most about 60 μm, at most about 50 μm, at most about 40 μm, at most about 30 μm, at most about 20 μm, at most about 10 μm, at most about 0 μm, or less. In some cases, the topical formulation can increase the thickness of the subject's nail by about 0 μm, about 10 μm, about 20 μm, about 30 μm, about 40 μm, about 50 μm, about 60 μm, about 70 μm, about 80 μm, about 90 μm, about 100 μm, about 200 μm, about 300 μm, about 400 μm, about 500 μm, about 600 μm, about 700 μm, about 800 μm, about 900 μm, about 1 mm, about 2 mm, about 3 mm, about 4 mm, about 5 mm, about 6 mm, about 7 mm, about 8 mm, about 9 mm, or about 10 mm.

In some embodiments, the topical formulation of the present disclosure can decrease a depth of ridges on the subject's nail. In some embodiments, the depth of ridges on the subject's nail can be measured by a Visioscan imaging system®. In some embodiments, the depth of ridges on the subject's nail can be measured by a Visioline® imaging system.

In some cases, the topical formulation can decrease a depth of vertical ridges on the subject's nail. In some cases, the topical formulation can decrease the depth of vertical ridges on the subject's nail compared to a baseline depth of vertical ridges on the subject's nail from a time point when the subject was not using the topical formulation. In some cases, the topical formulation can decrease the depth of the vertical ridges on the subject's nail by between about 0% to about 500%. In some cases, the topical formulation decreases the depth of vertical ridges on the subject's nail by between about 0% to about 500%. In some cases, the topical formulation decreases the depth of vertical ridges on the subject's nail by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the depth of vertical ridges on the subject's nail by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the depth of vertical ridges on the subject's nail by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%. In some cases, the topical formulation can decrease the depth of vertical ridges on the subject's nail by between about 0 nanometers (nm) to about 10 mm. In some cases, the topical formulation decreases the depth of vertical ridges on the subject's nail by at least about 0 nm, at least about 100 nm, at least about 200 nm, at least about 300 nm, at least about 400 nm, at least about 500 nm, at least about 600 nm, at least about 700 nm, at least about 800 nm, at least about 900 nm, at least about 1 μm, at least about 100 μm, at least about 200 μm, at least about 300 μm, at least about 400 μm, at least about 500 μm, at least about 600 μm, at least about 700 μm, at least about 800 μm, at least about 900 μm, at least about 1 mm, at least about 2 mm, at least about 3 mm, at least about 4 mm, at least about 5 mm, at least about 6 mm, at least about 7 mm, at least about 8 mm, at least about 9 mm, at least about 10 mm, or more. In some cases, the topical formulation decrease the depth of the vertical ridges on the subject's nail by at most about 10 mm, at most about 9 mm, at most about 8 mm, at most about 7 mm, at most about 6 mm, at most about 5 mm, at most about 4 mm, at most about 3 mm, at most about 2 mm, at most about 1 mm, at most about 900 μm, at most about 800 μm, at most about 700 μm, at most about 600 μm, at most about 500 μm, at most about 400 μm, at most about 300 μm, at most about 200 μm, at most about 100 μm, at most about 1 μm, at most about 900 nm, at most about 800 nm, at most about 700 nm, at most about 600 nm, at most about 500 nm, at most about 400 nm, at most about 300 nm, at most about 200 nm, at most about 100 nm, at most about 0 nm, or less. In some cases, the topical formulation decreases the depth of vertical ridges on the subject's nail by about 0 nm, about 100 nm, about 200 nm, about 300 nm, about 400 nm, about 500 nm, about 600 nm, about 700 nm, about 800 nm, about 900 nm, about 1 μm, about 100 μm, about 200 μm, about 300 μm, about 400 μm, about 500 μm, about 600 μm, about 700 μm, about 800 μm, about 900 μm, about 1 mm, about 2 mm, about 3 mm, about 4 mm, about 5 mm, about 6 mm, about 7 mm, about 8 mm, about 9 mm, or about 10 mm.

In some cases, the topical formulation can decrease a depth of horizontal ridges on the subject's nail. In some cases, the topical formulation can decrease the depth of horizontal ridges on the subject's nail compared to a baseline depth of horizontal ridges on the subject's nail from a time point when the subject was not using the topical formulation. In some cases, the topical formulation can decrease the depth of the horizontal ridges on the subject's nail by between about 0% to about 500%. In some cases, the topical formulation decreases the depth of horizontal ridges on the subject's nail by between about 0% to about 500%. In some cases, the topical formulation decreases the depth of horizontal ridges on the subject's nail by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the depth of horizontal ridges on the subject's nail by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the depth of horizontal ridges on the subject's nail by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%. In some cases, the topical formulation can decrease the depth of horizontal ridges on the subject's nail by between about 0 nanometers (nm) to about 10 mm. In some cases, the topical formulation decreases the depth of horizontal ridges on the subject's nail by at least about 0 nm, at least about 100 nm, at least about 200 nm, at least about 300 nm, at least about 400 nm, at least about 500 nm, at least about 600 nm, at least about 700 nm, at least about 800 nm, at least about 900 nm, at least about 1 μm, at least about 100 μm, at least about 200 μm, at least about 300 μm, at least about 400 μm, at least about 500 μm, at least about 600 μm, at least about 700 μm, at least about 800 μm, at least about 900 μm, at least about 1 mm, at least about 2 mm, at least about 3 mm, at least about 4 mm, at least about 5 mm, at least about 6 mm, at least about 7 mm, at least about 8 mm, at least about 9 mm, at least about 10 mm, or more. In some cases, the topical formulation decrease the depth of the horizontal ridges on the subject's nail by at most about 10 mm, at most about 9 mm, at most about 8 mm, at most about 7 mm, at most about 6 mm, at most about 5 mm, at most about 4 mm, at most about 3 mm, at most about 2 mm, at most about 1 mm, at most about 900 μm, at most about 800 μm, at most about 700 μm, at most about 600 μm, at most about 500 μm, at most about 400 μm, at most about 300 μm, at most about 200 μm, at most about 100 μm, at most about 1 μm, at most about 900 nm, at most about 800 nm, at most about 700 nm, at most about 600 nm, at most about 500 nm, at most about 400 nm, at most about 300 nm, at most about 200 nm, at most about 100 nm, at most about 0 nm, or less. In some cases, the topical formulation decreases the depth of horizontal ridges on the subject's nail by about 0 nm, about 100 nm, about 200 nm, about 300 nm, about 400 nm, about 500 nm, about 600 nm, about 700 nm, about 800 nm, about 900 nm, about 1 μm, about 100 μm, about 200 μm, about 300 μm, about 400 μm, about 500 μm, about 600 μm, about 700 μm, about 800 μm, about 900 μm, about 1 mm, about 2 mm, about 3 mm, about 4 mm, about 5 mm, about 6 mm, about 7 mm, about 8 mm, about 9 mm, or about 10 mm.

In some cases, the topical formulation can decrease a depth of horizontal ridges and a depth of vertical ridges on the subject's nail. In some cases, the topical formulation can decrease the depth of vertical ridges and the depth of horizontal ridges on the subject's nail compared to a baseline depth of vertical ridges and a baseline depth of horizontal ridges on the subject's nail from a time point when the subject was not using the topical formulation. In some cases, the topical formulation can decrease the depth of the horizontal ridges and the depth of the vertical ridges on the subject's nail by between about 0% to about 500%. In some cases, the topical formulation decreases the depth of horizontal ridges and the depth of vertical ridges on the subject's nail by between about 0% to about 500%. In some cases, the topical formulation decreases the depth of horizontal ridges and the depth of vertical ridges on the subject's nail by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the depth of horizontal ridges and the depth of vertical ridges on the subject's nail by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the depth of horizontal ridges and the depth of vertical ridges on the subject's nail by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%. In some cases, the topical formulation can decrease the depth of horizontal ridges and the depth of vertical ridges on the subject's nail by between about 0 nanometers (nm) to about 10 mm. In some cases, the topical formulation decreases the depth of horizontal ridges and the depth of vertical ridges on the subject's nail by at least about 0 nm, at least about 100 nm, at least about 200 nm, at least about 300 nm, at least about 400 nm, at least about 500 nm, at least about 600 nm, at least about 700 nm, at least about 800 nm, at least about 900 nm, at least about 1 μm, at least about 100 μm, at least about 200 μm, at least about 300 μm, at least about 400 μm, at least about 500 μm, at least about 600 μm, at least about 700 μm, at least about 800 μm, at least about 900 μm, at least about 1 mm, at least about 2 mm, at least about 3 mm, at least about 4 mm, at least about 5 mm, at least about 6 mm, at least about 7 mm, at least about 8 mm, at least about 9 mm, at least about 10 mm, or more. In some cases, the topical formulation decrease the depth of the horizontal ridges and the depth of the vertical ridges on the subject's nail by at most about 10 mm, at most about 9 mm, at most about 8 mm, at most about 7 mm, at most about 6 mm, at most about 5 mm, at most about 4 mm, at most about 3 mm, at most about 2 mm, at most about 1 mm, at most about 900 μm, at most about 800 μm, at most about 700 μm, at most about 600 μm, at most about 500 μm, at most about 400 μm, at most about 300 μm, at most about 200 μm, at most about 100 μm, at most about 1 μm, at most about 900 nm, at most about 800 nm, at most about 700 nm, at most about 600 nm, at most about 500 nm, at most about 400 nm, at most about 300 nm, at most about 200 nm, at most about 100 nm, at most about 0 nm, or less. In some cases, the topical formulation decreases the depth of horizontal ridges and the depth of vertical ridges on the subject's nail by about 0 nm, about 100 nm, about 200 nm, about 300 nm, about 400 nm, about 500 nm, about 600 nm, about 700 nm, about 800 nm, about 900 nm, about 1 μm, about 100 μm, about 200 μm, about 300 μm, about 400 μm, about 500 μm, about 600 μm, about 700 μm, about 800 μm, about 900 μm, about 1 mm, about 2 mm, about 3 mm, about 4 mm, about 5 mm, about 6 mm, about 7 mm, about 8 mm, about 9 mm, or about 10 mm.

In some embodiments, the topical formulation of the present disclosure can decrease a width of ridges on the subject's nail. In some embodiments, the width of ridges on the subject's nail can be measured by a Visioscan imaging system®. In some embodiments, the width of ridges on the subject's nail can be measured by a Visioline® imaging system.

In some cases, the topical formulation can decrease a width of vertical ridges on the subject's nail. In some cases, the topical formulation can decrease the width of vertical ridges on the subject's nail compared to a baseline width of vertical ridges on the subject's nail from a time point when the subject was not using the topical formulation. In some cases, the topical formulation can decrease the width of the vertical ridges on the subject's nail by between about 0% to about 500%. In some cases, the topical formulation decreases the width of vertical ridges on the subject's nail by between about 0% to about 500%. In some cases, the topical formulation decreases the width of vertical ridges on the subject's nail by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the width of horizontal ridges on the subject's nail by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the width of vertical ridges on the subject's nail by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%. In some cases, the topical formulation can decrease the width of vertical ridges on the subject's nail by between about 0 nanometers (nm) to about 10 mm. In some cases, the topical formulation decreases the width of vertical ridges on the subject's nail by at least about 0 nm, at least about 100 nm, at least about 200 nm, at least about 300 nm, at least about 400 nm, at least about 500 nm, at least about 600 nm, at least about 700 nm, at least about 800 nm, at least about 900 nm, at least about 1 μm, at least about 100 μm, at least about 200 μm, at least about 300 μm, at least about 400 μm, at least about 500 μm, at least about 600 μm, at least about 700 μm, at least about 800 μm, at least about 900 μm, at least about 1 mm, at least about 2 mm, at least about 3 mm, at least about 4 mm, at least about 5 mm, at least about 6 mm, at least about 7 mm, at least about 8 mm, at least about 9 mm, at least about 10 mm, or more. In some cases, the topical formulation decrease the width of vertical ridges on the subject's nail by at most about 10 mm, at most about 9 mm, at most about 8 mm, at most about 7 mm, at most about 6 mm, at most about 5 mm, at most about 4 mm, at most about 3 mm, at most about 2 mm, at most about 1 mm, at most about 900 μm, at most about 800 μm, at most about 700 μm, at most about 600 μm, at most about 500 μm, at most about 400 μm, at most about 300 μm, at most about 200 μm, at most about 100 μm, at most about 1 μm, at most about 900 nm, at most about 800 nm, at most about 700 nm, at most about 600 nm, at most about 500 nm, at most about 400 nm, at most about 300 nm, at most about 200 nm, at most about 100 nm, at most about 0 nm, or less. In some cases, the topical formulation decreases the width of vertical ridges on the subject's nail by about 0 nm, about 100 nm, about 200 nm, about 300 nm, about 400 nm, about 500 nm, about 600 nm, about 700 nm, about 800 nm, about 900 nm, about 1 μm, about 100 μm, about 200 μm, about 300 μm, about 400 μm, about 500 μm, about 600 μm, about 700 μm, about 800 μm, about 900 μm, about 1 mm, about 2 mm, about 3 mm, about 4 mm, about 5 mm, about 6 mm, about 7 mm, about 8 mm, about 9 mm, or about 10 mm.

In some cases, the topical formulation can decrease a width of horizontal ridges on the subject's nail. In some cases, the topical formulation can decrease the width of horizontal ridges on the subject's nail compared to a baseline width of horizontal ridges on the subject's nail from a time point when the subject was not using the topical formulation. In some cases, the topical formulation can decrease the width of the horizontal ridges on the subject's nail by between about 0% to about 500%. In some cases, the topical formulation decreases the width of horizontal ridges on the subject's nail by between about 0% to about 500%. In some cases, the topical formulation decreases the width of horizontal ridges on the subject's nail by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the width of horizontal ridges on the subject's nail by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the width of horizontal ridges on the subject's nail by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%. In some cases, the topical formulation can decrease the width of horizontal ridges on the subject's nail by between about 0 nanometers (nm) to about 10 mm. In some cases, the topical formulation decreases the width of horizontal ridges on the subject's nail by at least about 0 nm, at least about 100 nm, at least about 200 nm, at least about 300 nm, at least about 400 nm, at least about 500 nm, at least about 600 nm, at least about 700 nm, at least about 800 nm, at least about 900 nm, at least about 1 μm, at least about 100 μm, at least about 200 μm, at least about 300 μm, at least about 400 μm, at least about 500 μm, at least about 600 μm, at least about 700 μm, at least about 800 μm, at least about 900 μm, at least about 1 mm, at least about 2 mm, at least about 3 mm, at least about 4 mm, at least about 5 mm, at least about 6 mm, at least about 7 mm, at least about 8 mm, at least about 9 mm, at least about 10 mm, or more. In some cases, the topical formulation decrease the width of the horizontal ridges on the subject's nail by at most about 10 mm, at most about 9 mm, at most about 8 mm, at most about 7 mm, at most about 6 mm, at most about 5 mm, at most about 4 mm, at most about 3 mm, at most about 2 mm, at most about 1 mm, at most about 900 μm, at most about 800 μm, at most about 700 μm, at most about 600 μm, at most about 500 μm, at most about 400 μm, at most about 300 μm, at most about 200 μm, at most about 100 μm, at most about 1 μm, at most about 900 nm, at most about 800 nm, at most about 700 nm, at most about 600 nm, at most about 500 nm, at most about 400 nm, at most about 300 nm, at most about 200 nm, at most about 100 nm, at most about 0 nm, or less. In some cases, the topical formulation decreases the width of horizontal ridges on the subject's nail by about 0 nm, about 100 nm, about 200 nm, about 300 nm, about 400 nm, about 500 nm, about 600 nm, about 700 nm, about 800 nm, about 900 nm, about 1 μm, about 100 μm, about 200 μm, about 300 μm, about 400 μm, about 500 μm, about 600 μm, about 700 μm, about 800 μm, about 900 μm, about 1 mm, about 2 mm, about 3 mm, about 4 mm, about 5 mm, about 6 mm, about 7 mm, about 8 mm, about 9 mm, or about 10 mm.

In some cases, the topical formulation can decrease a width of vertical ridges and a width of horizontal ridges on the subject's nail. In some cases, the topical formulation can decrease the width of horizontal ridges and the width of vertical ridges on the subject's nail compared to a baseline width of horizontal ridges and a baseline width of vertical ridges on the subject's nail from a time point when the subject was not using the topical formulation. In some cases, the topical formulation can decrease the width of the horizontal ridges and the width of the vertical ridges on the subject's nail by between about 0% to about 500%. In some cases, the topical formulation decreases the width of horizontal ridges and the width of vertical ridges on the subject's nail by between about 0% to about 500%. In some cases, the topical formulation decreases the width of horizontal ridges and the width of vertical ridges on the subject's nail by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the width of horizontal ridges and the width of vertical ridges on the subject's nail by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the width of horizontal ridges and the width of vertical ridges on the subject's nail by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%. In some cases, the topical formulation can decrease the width of horizontal ridges and the width of vertical ridges on the subject's nail by between about 0 nanometers (nm) to about 10 mm. In some cases, the topical formulation decreases the width of horizontal ridges and the width of vertical ridges on the subject's nail by at least about 0 nm, at least about 100 nm, at least about 200 nm, at least about 300 nm, at least about 400 nm, at least about 500 nm, at least about 600 nm, at least about 700 nm, at least about 800 nm, at least about 900 nm, at least about 1 μm, at least about 100 μm, at least about 200 μm, at least about 300 μm, at least about 400 μm, at least about 500 μm, at least about 600 μm, at least about 700 μm, at least about 800 μm, at least about 900 μm, at least about 1 mm, at least about 2 mm, at least about 3 mm, at least about 4 mm, at least about 5 mm, at least about 6 mm, at least about 7 mm, at least about 8 mm, at least about 9 mm, at least about 10 mm, or more. In some cases, the topical formulation decrease the width of the horizontal ridges and the width of the vertical ridges on the subject's nail by at most about 10 mm, at most about 9 mm, at most about 8 mm, at most about 7 mm, at most about 6 mm, at most about 5 mm, at most about 4 mm, at most about 3 mm, at most about 2 mm, at most about 1 mm, at most about 900 μm, at most about 800 μm, at most about 700 μm, at most about 600 μm, at most about 500 μm, at most about 400 μm, at most about 300 μm, at most about 200 μm, at most about 100 μm, at most about 1 μm, at most about 900 nm, at most about 800 nm, at most about 700 nm, at most about 600 nm, at most about 500 nm, at most about 400 nm, at most about 300 nm, at most about 200 nm, at most about 100 nm, at most about 0 nm, or less. In some cases, the topical formulation decreases the width of horizontal ridges and the width of vertical ridges on the subject's nail by about 0 nm, about 100 nm, about 200 nm, about 300 nm, about 400 nm, about 500 nm, about 600 nm, about 700 nm, about 800 nm, about 900 nm, about 1 μm, about 100 μm, about 200 μm, about 300 μm, about 400 μm, about 500 μm, about 600 μm, about 700 μm, about 800 μm, about 900 μm, about 1 mm, about 2 mm, about 3 mm, about 4 mm, about 5 mm, about 6 mm, about 7 mm, about 8 mm, about 9 mm, or about 10 mm.

In some embodiments, the topical formulation of the present disclosure can decrease an overall number of ridges (e.g., vertical ridge and horizontal ridges) on the subject's nail. In some embodiments, the overall number of ridges on the subject's nail can be measured by a Visioscan imaging system®. In some embodiments, the overall number of ridges on the subject's nail can be measured by a Visioline® imaging system.

In some cases, the topical formulation of the present disclosure can decrease an overall number of vertical ridges on the subject's nail. In some cases, the topical formulation can decrease the overall number of vertical ridges on the subject's nail compared to a baseline overall number of vertical ridges on the subject's nail from a time point when the subject was not using the topical formulation. In some cases, the topical formulation can decrease the overall number of vertical ridges on the subject's nail by between about 0% to about 500%. In some cases, the topical formulation decreases the overall number of vertical ridges on the subject's nail by between about 0% to about 500%. In some cases, the topical formulation decreases the overall number of vertical ridges on the subject's nail by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the overall number of vertical ridges on the subject's nail by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the overall number of vertical ridges on the subject's nail by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some cases, the topical formulation of the present disclosure can decrease an overall number of horizontal ridges on the subject's nail. In some cases, the topical formulation can decrease the overall number of horizontal ridges on the subject's nail compared to a baseline overall number of horizontal ridges on the subject's nail from a time point when the subject was not using the topical formulation. In some cases, the topical formulation can decrease the overall number the horizontal ridges on the subject's nail by between about 0% to about 500%. In some cases, the topical formulation decreases the overall number of horizontal ridges on the subject's nail by between about 0% to about 500%. In some cases, the topical formulation decreases the overall number of horizontal ridges on the subject's nail by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the overall number of horizontal ridges on the subject's nail by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the overall number of horizontal ridges on the subject's nail by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some cases, the topical formulation of the present disclosure can decrease an overall number of vertical ridges and an overall number of horizontal ridges on the subject's nail. In some cases, the topical formulation can decrease the overall number of horizontal ridges and the overall number of vertical ridges on the subject's nail compared to a baseline overall number of horizontal ridges and a baseline overall number of vertical ridges on the subject's nail from a time point when the subject was not using the topical formulation. In some cases, the topical formulation can decrease the overall number of horizontal ridges and the overall number of vertical ridges on the subject's nail by between about 0% to about 500%. In some cases, the topical formulation decreases the overall number of horizontal ridges and the overall number of vertical ridges on the subject's nail by between about 0% to about 500%. In some cases, the topical formulation decreases the overall number of horizontal ridges and the overall number of vertical ridges on the subject's nail by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the overall number of horizontal ridges and the overall number of vertical ridges on the subject's nail by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the overall number of horizontal ridges and the overall number of vertical ridges on the subject's nail by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation of the present disclosure can increase a smoothness of the subject's nail. In some embodiments, the smoothness of the subject's nail can be measured by a Visioscan imaging system®. In some embodiments, the smoothness of the subject's nail can be measured by a Visioline® imaging system.

In some cases, the topical formulation of the present disclosure can increase the smoothness of the subject's nail. In some cases, the topical formulation increases the smoothness of the subject's nail compared to a baseline smoothness of the subject's nail from a time point when the subject was not using the topical formulation. In some cases, the topical formulation increases the smoothness of the subject's nail by between about 0% to about 500%. In some cases, the topical formulation increases the smoothness of the subject's nail by between about 0% to about 500%. In some cases, the topical formulation increases the smoothness of the subject's nail by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure increases the smoothness of the subject's nail by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure increases the smoothness of the subject's nail by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%. In some embodiments, the topical formulation increases the smoothness of the subject's nail by at least about 92%.

In some embodiments, the topical formulation of the present disclosure can increase a nail plate hardness of the subject's nail. The nail plate can be a portion of the nail that is parallel to surface of the subject's finger or toe. In some cases, the nail plate hardness of the subject's nail can be measured by a durometer. In some cases, the nail plate hardness of the subject's nail can be measured by a Knoop indenter. In some cases, the nail plate hardness of the subject's nail can be measured by onychoscopy.

In some cases, the topical formulation increases the nail plate hardness of the subject's nail compared to a baseline nail plate hardness of the subject's nail from a time point when the subject was not using the topical formulation. In some cases, the topical formulation increases the nail plate hardness of the subject's nail by between about 0% to about 500%. In some cases, the topical formulation increases the nail plate hardness of the subject's nail by between about 0% to about 500%. In some cases, the topical formulation increases the nail plate hardness of the subject's nail by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure increases the nail plate hardness of the subject's nail by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure increases the nail plate hardness of the subject's nail by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation of the present disclosure can increase a nail fold hardness of the subject's nail. The nail fold can be a portion of the nail that is perpendicular to surface of the subject's finger or toe. In some cases, the nail fold hardness of the subject's nail can be measured by a durometer. In some cases, the nail fold hardness of the subject's nail can be measured by a Knoop indenter. In some cases, the nail fold hardness of the subject's nail can be measured by onychoscopy.

In some cases, the topical formulation increases the nail fold hardness of the subject's nail compared to a baseline nail fold hardness of the subject's nail from a time point when the subject was not using the topical formulation. In some cases, the topical formulation increases the nail fold hardness of the subject's nail by between about 0% to about 500%. In some cases, the topical formulation increases the nail fold hardness of the subject's nail by between about 0% to about 500%. In some cases, the topical formulation increases the nail fold hardness of the subject's nail by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure increases the nail fold hardness of the subject's nail by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure increases the nail fold hardness of the subject's nail by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation of the present disclosure can increase nail strength. In some embodiments, the topical formulation of the present disclosure can increase nail strength by between about 0% to about 500%. In some cases, the topical formulation increases the nail fold hardness of the subject's nail by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure increases the nail fold hardness of the subject's nail by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure increases the nail fold hardness of the subject's nail by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%. In some embodiments, the topical formulation increases the nail fold hardness of the subject's nail by at least about 54%.

In some embodiments, the topical formulation of the present disclosure can decrease the nail free edge of the subject's nail. The nail free edge thickness can be the thickness of an edge of the nail not directly connected to the subject's finger or toe. In some cases, the nail free edge thickness can be measured from a clipping taken from the nail free edge. In some cases, the nail free edge thickness can be measured from the nail free edge attached to the nail plate of the subject's nail. In some cases, the nail free edge can be white, translucent, yellow, or any combination thereof in appearance. In some cases, the nail free edge thickness can be measured by measuring the thickness of the nail clipping taken from the nail free edge.

In some cases, the topical formulation can decrease the nail free edge thickness of the subject's nail compared to a baseline nail free edge thickness of the subject's nail from a time point when the subject was not using the topical formulation. In some cases, the topical formulation can decrease the nail free edge thickness of the subject's nail by between about 0% to about 500%. In some cases, the topical formulation decreases the nail free edge thickness of the subject's nail by between about 0% to about 500%. In some cases, the topical formulation decreases the nail free edge thickness of the subject's nail by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure decreases the nail free edge thickness of the subject's nail by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure decreases the nail free edge thickness of the subject's nail by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%. In some cases, the topical formulation can decrease the nail free edge thickness of the subject's nail by between about 0 nanometers (nm) to about 10 mm. In some cases, the topical formulation decreases the nail free edge thickness of the subject's nail by at least about 0 nm, at least about 100 nm, at least about 200 nm, at least about 300 nm, at least about 400 nm, at least about 500 nm, at least about 600 nm, at least about 700 nm, at least about 800 nm, at least about 900 nm, at least about 1 μm, at least about 100 μm, at least about 200 μm, at least about 300 μm, at least about 400 μm, at least about 500 μm, at least about 600 μm, at least about 700 μm, at least about 800 μm, at least about 900 μm, at least about 1 mm, at least about 2 mm, at least about 3 mm, at least about 4 mm, at least about 5 mm, at least about 6 mm, at least about 7 mm, at least about 8 mm, at least about 9 mm, at least about 10 mm, or more. In some cases, the topical formulation decreases the nail free edge thickness of the subject's nail by at most about 10 mm, at most about 9 mm, at most about 8 mm, at most about 7 mm, at most about 6 mm, at most about 5 mm, at most about 4 mm, at most about 3 mm, at most about 2 mm, at most about 1 mm, at most about 900 μm, at most about 800 μm, at most about 700 μm, at most about 600 μm, at most about 500 μm, at most about 400 μm, at most about 300 μm, at most about 200 μm, at most about 100 μm, at most about 1 μm, at most about 900 nm, at most about 800 nm, at most about 700 nm, at most about 600 nm, at most about 500 nm, at most about 400 nm, at most about 300 nm, at most about 200 nm, at most about 100 nm, at most about 0 nm, or less. In some cases, the topical formulation decreases the nail free edge thickness of the subject's nail by about 0 nm, about 100 nm, about 200 nm, about 300 nm, about 400 nm, about 500 nm, about 600 nm, about 700 nm, about 800 nm, about 900 nm, about 1 μm, about 100 μm, about 200 μm, about 300 μm, about 400 μm, about 500 μm, about 600 μm, about 700 μm, about 800 μm, about 900 μm, about 1 mm, about 2 mm, about 3 mm, about 4 mm, about 5 mm, about 6 mm, about 7 mm, about 8 mm, about 9 mm, or about 10 mm.

In some embodiments, the topical formulation of the present disclosure can increase a length of the nail free edge of the subject's nail. The length of the nail free edge can be measured by measuring a length of the nail free edge starting wherein the nail free edge is connected to the nail plate to the end of the nail. In some cases, the length of the nail free edge can be measured via a measuring tape or ruler. In some cases, the length of the nail free edge can be measured via photographs of the subject's nail. In some examples, the photographs of the subject's nail can be analyzed to determine the length of the nail free edge of the subject's nail. In some examples, the photographs of the subject's nail can be analyzed via PhotoGrammetrix® Image Analysis 3D Zephyr software, PhotoModeler software, Agisoft PhotoScan software, Metashape software, Pix4Dmapper software, or any combination thereof.

In some cases, the topical formulation can increase the length of the nail free edge of the subject's nail compared to a baseline length of the nail free edge of the subject's nail from a time point when the subject was not using the topical formulation. In some cases, the topical formulation increases the length of the nail free edge of the subject's nail by between about 0% to about 500%. In some cases, the topical formulation increases the length of the nail free edge of the subject's nail by between about 0% to about 500%. In some cases, the topical formulation increases the length of the nail free edge of the subject's nail by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure increases the length of the nail free edge of the subject's nail by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure increases the length of the nail free edge of the subject's nail by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%. In some cases, the topical formulation can increase the length of the nail free edge of the subject's nail by between about 0 nanometers (nm) to about 10 mm. In some cases, the topical formulation increases the length of the nail free edge of the subject's nail by at least about 0 nm, at least about 100 nm, at least about 200 nm, at least about 300 nm, at least about 400 nm, at least about 500 nm, at least about 600 nm, at least about 700 nm, at least about 800 nm, at least about 900 nm, at least about 1 μm, at least about 100 μm, at least about 200 μm, at least about 300 μm, at least about 400 μm, at least about 500 μm, at least about 600 μm, at least about 700 μm, at least about 800 μm, at least about 900 μm, at least about 1 mm, at least about 2 mm, at least about 3 mm, at least about 4 mm, at least about 5 mm, at least about 6 mm, at least about 7 mm, at least about 8 mm, at least about 9 mm, at least about 10 mm, or more. In some cases, the topical formulation increases the length of the nail free edge of the subject's nail by at most about 10 mm, at most about 9 mm, at most about 8 mm, at most about 7 mm, at most about 6 mm, at most about 5 mm, at most about 4 mm, at most about 3 mm, at most about 2 mm, at most about 1 mm, at most about 900 μm, at most about 800 μm, at most about 700 μm, at most about 600 μm, at most about 500 μm, at most about 400 μm, at most about 300 μm, at most about 200 μm, at most about 100 μm, at most about 1 μm, at most about 900 nm, at most about 800 nm, at most about 700 nm, at most about 600 nm, at most about 500 nm, at most about 400 nm, at most about 300 nm, at most about 200 nm, at most about 100 nm, at most about 0 nm, or less. In some cases, the topical formulation increases the length of the nail free edge of the subject's nail by about 0 nm, about 100 nm, about 200 nm, about 300 nm, about 400 nm, about 500 nm, about 600 nm, about 700 nm, about 800 nm, about 900 nm, about 1 μm, about 100 μm, about 200 μm, about 300 μm, about 400 μm, about 500 μm, about 600 μm, about 700 μm, about 800 μm, about 900 μm, about 1 mm, about 2 mm, about 3 mm, about 4 mm, about 5 mm, about 6 mm, about 7 mm, about 8 mm, about 9 mm, or about 10 mm.

In some embodiments, the topical formulation of the present disclosure can increase the hydration of a nail root of the subject's nail. The nail root can be the regions of the skin that are directly connected to the nail. In some embodiments, the hydration of the nail root can be measured by a nail blanch test. In some embodiments, the hydration of the nail root can be measured by spectral-resolved ellipsometry. In some embodiments, the hydration of the nail root can be measured by thermogravimetric analysis procedure. In some embodiments, the hydration of the nail root can be measured by a dermal phase meter. In some embodiments, the hydration of the nail root can be measured by a skin moisture meter.

In some embodiments, the topical formulation of the present disclosure can increase overall hydration in the nail root of the subject's nail compared to a baseline overall hydration in the nail root of the subject's nail from a time point when the subject was not using the topical formulation. In some embodiments, the topical formulation of the present disclosure increases the overall hydration in the nail root of the subject's nail by between about 0% to about 500%. In some cases, the topical formulation of the present disclosure increases the overall hydration in the nail root of the subject's nail by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure increases the overall hydration in the nail root of the subject's nail by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure increases the overall hydration in the nail root of the subject's nail by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation of the present disclosure can increase overall conductance in the nail root of the subject's nail compared to a baseline overall conductance in the nail root of the subject's nail from a time point when the subject was not using the topical formulation. In some embodiments, the topical formulation of the present disclosure increases the overall conductance in the nail root of the subject's nail by between about 0% to about 500%. In some cases, the topical formulation of the present disclosure increases the overall conductance in the nail root of the subject's nail by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure increases the overall conductance in the nail root of the subject's nails by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure increases the overall conductance in the nail root of the subject's nail by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation of the present disclosure can increase overall capacitance in the nail root of the subject's nail compared to a baseline overall capacitance in the nail root of the subject's nail from a time point when the subject was not using the topical formulation. In some embodiments, the topical formulation of the present disclosure increases the overall capacitance in the nail root of the subject's nail by between about 0% to about 500%. In some cases, the topical formulation of the present disclosure increases the overall capacitance in the nail root of the subject's nail by at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more. In some cases, the topical formulation of the present disclosure increases the overall capacitance in the nail root of the subject's nail by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less. In some cases, the topical formulation of the present disclosure increases the overall capacitance in the nail root of the subject's nail by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the topical formulation of the present disclosure can evince signs of improvements in nail appearance and/or health in at least about 1 days, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 31 days, at least about 32 days, at least about 33 days, at least about 34 days, at least about 35 days, at least about 36 days, at least about 37 days, at least about 38 days, at least about 39 days, at least about 40 days, at least about 41 days, at least about 42 days, at least about 43 days, at least about 44 days, at least about 45 days, at least about 46 days, at least about 47 days, at least about 48 days, at least about 49 days, at least about 50 days, at least about 51 days, at least about 52 days, at least about 53 days, at least about 54 days, at least about 55 days, at least about 56 days, at least about 57 days, at least about 58 days, at least about 59 days, at least about 60 days, at least about 61 days, at least about 62 days, at least about 63 days, at least about 64 days, at least about 65 days, at least about 66 days, at least about 67 days, at least about 68 days, at least about 69 days, at least about 70 days, at least about 71 days, at least about 72 days, at least about 73 days, at least about 74 days, at least about 75 days, at least about 76 days, at least about 77 days, at least about 78 days, at least about 79 days, at least about 80 days, at least about 81 days, at least about 82 days, at least about 83 days, at least about 84 days, at least about 85 days, at least about 86 days, at least about 87 days, at least about 88 days, at least about 89 days, at least about 90 days, at least about 91 days, at least about 92 days, at least about 93 days, at least about 94 days, at least about 95 days, at least about 96 days, at least about 97 days, at least about 98 days, at least about 99 days, or at least about 100 days. In some embodiments, the topical formulation of the present disclosure can evince signs of improvements in nail appearance and/or health in at most about 100 days, at most about 99 days, at most about 98 days, at most about 97 days, at most about 96 days, at most about 95 days, at most about 94 days, at most about 93 days, at most about 92 days, at most about 91 days, at most about 90 days, at most about 89 days, at most about 88 days, at most about 87 days, at most about 86 days, at most about 85 days, at most about 84 days, at most about 83 days, at most about 82 days, at most about 81 days, at most about 80 days, at most about 79 days, at most about 78 days, at most about 77 days, at most about 76 days, at most about 75 days, at most about 74 days, at most about 73 days, at most about 72 days, at most about 71 days, at most about 70 days, at most about 69 days, at most about 68 days, at most about 67 days, at most about 66 days, at most about 65 days, at most about 64 days, at most about 63 days, at most about 62 days, at most about 61 days, at most about 60 days, at most about 59 days, at most about 58 days, at most about 57 days, at most about 56 days, at most about 55 days, at most about 54 days, at most about 53 days, at most about 52 days, at most about 51 days, at most about 50 days, at most about 49 days, at most about 48 days, at most about 47 days, at most about 46 days, at most about 45 days, at most about 44 days, at most about 43 days, at most about 42 days, at most about 41 days, at most about 40 days, at most about 39 days, at most about 38 days, at most about 37 days, at most about 36 days, at most about 35 days, at most about 34 days, at most about 33 days, at most about 32 days, at most about 31 days, at most about 30 days, at most about 29 days, at most about 28 days, at most about 27 days, at most about 26 days, at most about 25 days, at most about 24 days, at most about 23 days, at most about 22 days, at most about 21 days, at most about 20 days, at most about 19 days, at most about 18 days, at most about 17 days, at most about 16 days, at most about 15 days, at most about 14 days, at most about 13 days, at most about 12 days, at most about 11 days, at most about 10 days, at most about 9 days, at most about 8 days, at most about 7 days, at most about 6 days, at most about 5 days, at most about 4 days, at most about 3 days, at most about 2 days, at most about 1 day. In some embodiments, the topical formulation of the present disclosure can evince signs of improvements in nail appearance and/or health in about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, about 35 days, about 36 days, about 37 days, about 38 days, about 39 days, about 40 days, about 41 days, about 42 days, about 43 days, about 44 days, about 45 days, about 46 days, about 47 days, about 48 days, about 49 days, about 50 days, about 51 days, about 52 days, about 53 days, about 54 days, about 55 days, about 56 days, about 57 days, about 58 days, about 59 days, about 60 days, about 61 days, about 62 days, about 63 days, about 64 days, about 65 days, about 66 days, about 67 days, about 68 days, about 69 days, about 70 days, about 71 days, about 72 days, about 73 days, about 74 days, about 75 days, about 76 days, about 77 days, about 78 days, about 79 days, about 80 days, about 81 days, about 82 days, about 83 days, about 84 days, about 85 days, about 86 days, about 87 days, about 88 days, about 89 days, about 90 days, about 91 days, about 92 days, about 93 days, about 94 days, about 95 days, about 96 days, about 97 days, about 98 days, about 99 days, or about 100 days.

In some embodiments, the nail health improvements of the topical formulation of the present disclosure can last for at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 3 months, at least about 4 months, at least about 5 months, at least about 6 months, at least about 7 months, at least about 8 months, at least about 9 months, or at least about 10 months. In some embodiments, the nail health improvements of the topical formulation of the present disclosure can last for at most about 10 months, at most about 9 months, at most about 8 months, at most about 7 months, at most about 6 months, at most about 5 months, at most about 4 months, at most about 3 months, at most about 12 weeks, at most about 11 weeks, at most about 10 weeks, at most about 9 weeks, at most about 8 weeks, at most about 7 weeks, at most about 6 weeks, at most about 5 weeks, at most about 4 weeks, at most about 3 weeks, at most about 2 weeks, or at most about 1 week. In some embodiments, the nail health improvements of the topical formulation of the present disclosure can last for about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 3 months, about 4 months, about 5 months, about 6 months, about 7 months, about 8 months, about 9 months, or about 10 months.

### Other Ingredients

In some embodiments, the composition can have one or more cosmetically acceptable ingredients. In some embodiments, the one or more cosmetically acceptable ingredient can be Leuconostoc/radish root ferment filtrate, albumin, l-alanyl-l-glutamine, phospholipids, tocopheryl acetate, ubiquinone, Caesalpinia spinosa fruit extract, Helianthus annuus sprout extract, Pistacia lentiscus gum, squalane, panthenol, biotin, niacinamide, d-l methionine, Undaria Pinnatifida extract, Kappaphycus alvarezii extract, Ceratonia Siliqua fruit extract, Brassica alba sprout extract, or any combinations thereof. In some embodiments, the one or more cosmetically acceptable ingredients can be Leuconostoc/radish root ferment filtrate. In some embodiments, the one or more cosmetically acceptable ingredients can be albumin. In some embodiments, the one or more cosmetically acceptable ingredients can be 1-alanyl-l-glutamine. In some embodiments, the one or more cosmetically acceptable ingredients can be phospholipids. In some embodiments, the one or more cosmetically acceptable ingredients can be tocopheryl acetate. In some embodiments, the one or more cosmetically acceptable ingredients can be ubiquinone. In some embodiments, the one or more cosmetically acceptable ingredients can be Caesalpinia spinosa fruit extract. In some embodiments, the one or more cosmetically acceptable ingredients can be Caesalpinia spinosa fruit pod extract. In some embodiments, the one or more cosmetically acceptable ingredients can be Helianthus annuus sprout (Sunflower) extract. In some embodiments, the one or more cosmetically acceptable ingredients can be Pistacia lentiscus gum. In some embodiments, the one or more cosmetically acceptable ingredients can be squalene. In some embodiments, the one or more cosmetically acceptable ingredients can be panthenol. In some embodiments, the one or more cosmetically acceptable ingredients can be biotin. In some embodiments, the one or more cosmetically acceptable ingredients can be niacinamide. In some embodiments, the one or more cosmetically acceptable ingredients can be d-l methionine. In some embodiments, the one or more cosmetically acceptable ingredients can be Undaria pinnatifida extract. In some embodiments, the one or more cosmetically acceptable ingredients can be Caesalpinia spinosa fruit extract. In some embodiments, the one or more cosmetically acceptable ingredients can be Kappaphycus alvarezii extract. In some embodiments, the one or more cosmetically acceptable ingredients can be ceratonia siliqua fruit extract. In some embodiments, the one or more cosmetically acceptable ingredients can be brassica alba sprout extract. In some embodiments, the one or more cosmetically acceptable ingredients can be caprylic/capric triglycerides. In some embodiments, the one or more cosmetically acceptable ingredients can be water. In some embodiments, the one or more cosmetically acceptable ingredients can be Ricinus Communis (Castor) seed oil. In some embodiments, the one or more cosmetically acceptable ingredients can be bis-diglyceryl polyaceyladipate-2. In some embodiments, the one or more cosmetically acceptable ingredients can be rapeseed oil. In some cases, the rapeseed oil can be hydrogenated rapeseed oil. In some cases, the rapeseed oil can be non-hydrogenated rapeseed oil. In some embodiments, the one or more cosmetically acceptable ingredients can be ethylhexyl palmitate. In some embodiments, the one or more cosmetically acceptable ingredients can be Butyrospermum parkii shea) butter. In some embodiments, the one or more cosmetically acceptable ingredients can be Stearalkonium hectorite. In some embodiments, the one or more cosmetically acceptable ingredients can be Pyrus malus (apple) fruit extract. In some embodiments, the one or more cosmetically acceptable ingredients can be glycerin. In some embodiments, the one or more cosmetically acceptable ingredients can be Sambucus nigra fruit extract. In some embodiments, the one or more cosmetically acceptable ingredients can be lauroyl lysine. In some embodiments, the one or more cosmetically acceptable ingredients can be Glycine soja (soybean) seed extract. In some embodiments, the one or more cosmetically acceptable ingredients can be propylene carbonate. In some embodiments, the one or more cosmetically acceptable ingredients can be tocopheryl nicotinate. In some embodiments, the one or more cosmetically acceptable ingredients can be tetrahexyldecyl ascorbate. In some embodiments, the one or more cosmetically acceptable ingredients can be sodium hyaluronate. In some embodiments, the one or more cosmetically acceptable ingredients can be Undaria Pinnatifida extract. In some embodiments, the one or more cosmetically acceptable ingredients can be palmitoyl tripeptide-1. In some embodiments, the one or more cosmetically acceptable ingredients can be Epigallocatechin Gallatyl glucoside. In some embodiments, the one or more cosmetically acceptable ingredients can be Spirodela polyrhiza extract. In some embodiments, the one or more cosmetically acceptable ingredients can be bisabolol. In some embodiments, the one or more cosmetically acceptable ingredients can be panthenol. In some embodiments, the one or more cosmetically acceptable ingredients can be tocopheryl acetate. In some embodiments, the one or more cosmetically acceptable ingredients can be Glycyrrhiza glabra (licorice) leaf extract. In some embodiments, the one or more cosmetically acceptable ingredients can be Citrus limon (lemon) peel extract. In some embodiments, the one or more cosmetically acceptable ingredients can be polyglyceryl-2 dipolyhydroxystearate. In some embodiments, the one or more cosmetically acceptable ingredients can be Glucomannan. In some embodiments, the one or more cosmetically acceptable ingredients can be trihydroxystearin. In some embodiments, the one or more cosmetically acceptable ingredients can be tribehenin. In some embodiments, the one or more cosmetically acceptable ingredients can be sorbitan isostearate. In some embodiments, the one or more cosmetically acceptable ingredients can be potassium sorbate. In some embodiments, the one or more cosmetically acceptable ingredients can be sodium benzoate. In some embodiments, the one or more cosmetically acceptable ingredients can be citric acid. In some embodiments, the one or more cosmetically acceptable ingredients can be sodium chloride. In some embodiments, the one or more cosmetically acceptable ingredients can be pentylene glycol. In some embodiments, the one or more cosmetically acceptable ingredients can be octyldodecanol. In some embodiments, the one or more cosmetically acceptable ingredients can be glyceryl stearate citrate. In some embodiments, the one or more cosmetically acceptable ingredients can be methionine. In some embodiments, the one or more cosmetically acceptable ingredients can be polyglyceryl-3 stearate. In some embodiments, the one or more cosmetically acceptable ingredients can be lecithin. In some cases, the lecithin can be hydrogenated lecithin. In some cases, the lecithin can be non-hydrogenated lecithin. In some embodiments, the one or more cosmetically acceptable ingredients can be phytic acid. In some embodiments, the one or more cosmetically acceptable ingredients can be Pistacia lentiscus gum. In some embodiments, the one or more cosmetically acceptable ingredients can be Ribes Nigrum seed oil. In some embodiments, the one or more cosmetically acceptable ingredients can be Helianthus annuus seed oil unsaponifiables. In some embodiments, the one or more cosmetically acceptable ingredients can be Cardiospermum halicacabum flower extract. In some embodiments, the one or more cosmetically acceptable ingredients can be Cardiospermum halicacabum leaf extract. In some embodiments, the one or more cosmetically acceptable ingredients can be Cardiospermum halicacabum vine extract. In some embodiments, the one or more cosmetically acceptable ingredients can be tocopherol. In some embodiments, the one or more cosmetically acceptable ingredients can be Helianthus annuus seed oil. In some embodiments, the one or more cosmetically acceptable ingredients can be Rosmarinus Officinalis lead extract. In some embodiments, the one or more cosmetically acceptable ingredients can be epigallocatechin gallatyl glucoside. In some embodiments, the one or more cosmetically acceptable ingredients can be propylene glycol. In some embodiments, the one or more cosmetically acceptable ingredients can be polyacrylate crosspolymer-6. In some embodiments, the one or more cosmetically acceptable ingredients can be phenoxyethanol. In some embodiments, the topical formulate can comprise any combination of the one or more cosmetically acceptable ingredients disclosed herein.

In some embodiments, any formulation as described herein can comprise defensins, natural molecules that can assist regenerative cells. In some embodiments, any formulation as described herein can comprise methionine, an essential amino acid. In some embodiments, any formulation as described herein can comprise tara tree fruit extract, an antioxidant that can protect nails and keratin structures from damaging free radicals caused by environmental stress, UV light, and/or harsh chemicals. In some embodiments, any formulation as described herein can comprise a vitamin B complex comprising Vitamin B3 (niacinamide), Vitamin B5 (panthenol), and Vitamin B7 (biotin), which can repair natural moisturization of skin and keratin. In some embodiments, any formulation as described herein can comprise a blend of soothing and/or calming ingredients comprising flower extracts, epigallocatechin gallatyl glucoside, and black currant extract.

In some embodiments, a formulation as described herein comprises: water, panthenol, niacinamide, pentylene glycol, squalane, Ricinus communis (castor) seed oil, octyldodecanol, glyceryl stearate citrate, alpha-defensin 5, beta-defensin 3, biotin, Caesalpinia spinosa fruit pod extract/Caesalpinia spinosa fruit extract, Cardiospermum halicacabum flower/leaf/vine extract, Helianthus annuus (sunflower) sprout extract, Helianthus annuus seed oil unsaponifiables, Helianthus annuus seed oil, Ribes nigrum seed oil, Rosmarinus officinalis leaf extract, Leuconostoc/radish root ferment filtrate, methionine, epigallocatechin gallatyl glucoside, phospholipids, tocopherol, tocopheryl acetate, ubiquinone, l-alanyl-l-glutamine, polyglyceryl-3 stearate, hydrogenated lecithin, phytic acid, caprylic/capric triglyceride, Pistacia lentiscus gum, polyacrylate crosspolymer-6, phenoxyethanol, albumin, sodium benzoate, potassium sorbate, propylene glycol, sodium chloride, or any combination thereof. In some embodiments, any formulation as described herein can be fragrance free. In some embodiments, any formulation as described herein can have a fragrance added.

In some embodiments, a formulation as described herein comprises the ingredients: caprylic/capric triglyceride, water, Ricinus communis (castor) seed oil, bis-diglyceryl polyacryladipate-2, hydrogenated rapeseed oil, ethylhexyl palmitate, Butyrospermum parkii (shea) butter, stearalkonium hectorite, Pyrus malus (apple) fruit extract, glycerin, niacinamide, Sambucus nigra fruit extract, lauroyl lysine, Glycine soja (soybean) seed extract, propylene carbonate, phospholipids, alpha-defensin 5 (sh-oligopeptide-109 SP), beta-defensin 3 (sh-oligopeptide-108 SP), ubiquinone, tocopheryl nicotinate, tetra hexyldecyl ascorbate, sodium hyaluronate, Undaria pinnatifida extract, palmitoyl tripeptide-1, Brassica alba sprout extract, epigallocatechin gallatyl glucoside, Caesalpinia spinosa fruit extract, Kappaphycus alvarezii extract, Spirodela polyrhiza extract, bisabolol, panthenol, tocopheryl acetate, Glycyrrhiza glabra (licorice) leaf extract, Citrus limon (lemon) peel extract, albumin, l-alanyl-l-glutamine, polyglyceryl-2 dipolyhydroxystearate, glucomannan, trihydroxystearin, tribehenin, sorbitan isostearate, potassium sorbate, Leuconostoc/radish root ferment filtrate, sodium benzoate, citric acid, sodium chloride, or any combination thereof. In some embodiments, any formulation as described herein can be fragrance free. In some embodiments, any formulation as described herein can have a fragrance added.

In some embodiments, the defensin can be associated with a cosmetically acceptable protein to increase stability and/or delivery characteristics. In some embodiments, the association can be non-covalent (e.g., electrostatic, ionic, hydrophobic, etc.). In some embodiments, the association can be covalent. Non-limiting examples of cosmetically acceptable proteins include lactoferrin, transferrin, and albumin (e.g., human serum albumin, bovine serum albumin, and egg albumin, recombinant albumin). The defensins and protein carriers may be in various ratios, including equimolar, sub-, and supramolar ratios. In some cases, combinations of two or more protein carriers may be used. In some examples, in a formulation in which two defensins are used, one defensin may be associated with one carrier, and the other defensin may be associated with a different carrier. In some embodiments, any combination of defensins and carriers are contemplated.

In some embodiments, the defensins can be encapsulated in a lipid membrane delivery system. Non-limiting examples of lipid membrane delivery system include liposomes, microcapsules, nanocapsules, microparticles, nanoparticles, and microparticle delivery systems. In some embodiments, the defensin can be encapsulated in a liposome. In some embodiments, the defensins can be encapsulated in microcapsules. In some embodiments, the defensins can be encapsulated in nanocapsules. In some embodiments, the defensins can be encapsulated in nanoparticles. In some embodiments, the defensins can be encapsulated in microparticles. A description of some cosmetically acceptable cosmetic delivery systems can be found in Maherani et al, “Liposomes: A Review of Manufacturing Techniques and Targeting Strategy,” Current Nanoscience; 7:436-452 (2011). In some embodiments, liposomes can be manufactured using the shear method. In some embodiments, lipid membrane delivery systems can resemble naturally occurring membranes. In some embodiments, the lipid membrane delivery system can be flexible. In some embodiments, the lipid membrane delivery systems can penetrate interstitial spaces between cells. In some embodiments, the lipid membrane delivery systems may have monolayer, bilayer (e.g., unilamellar vesicle or ULV), or multi-layer structures (e.g. multilammerlar vesicle or MLV). In some cases, multilayer lipid membrane delivery systems may have nested structures (e.g., multivesicular vesicle or MVV). In some embodiments, lipid membrane delivery systems used in the compositions can range in size from about 500 nm to about 10 microns. In some embodiments, the lipid membrane delivery system used in the composition can be at least about 500 nm, at least about 600 nm, at least about 700 nm, at least about 800 nm, at least about 900 nm, at least about 1 micromicron (um), at least about 100 μm, at least about 200 μm, at least about 300 μm, at least about 400 μm, at least about 500 μm, at least about 600 μm, at least about 700 μm, at least about 800 μm, at least about 900, at least 1 millimicron (mm), at least about 100 mm, at least about 200 mm, at least about 200 mm, at least about 300 mm, at least about 400 mm, at least about 500 mm, at least about 600 mm, at least about 700, at least about 800, at least about 900, at least about 1 micron, at least about 5 microns, at least about 10 microns, or more. In some embodiments, the lipid membrane delivery system used in the composition can be at most about 10 microns, at most about 5 microns, at most about 1 micron, at most about 900 mm, at most about 800 mm, at most about 700 mm, at most about 600 mm, at most about 500 mm, at most about 400 mm, at most about 300 mm, at most about 200 mm, at most about 100 mm, at most about 1 mm, at most about 900 μm, at most about 800 μm, at most about 700 μm, at most about 600 μm, at most about 500 μm, at most about 400 μm, at most about 300 μm, at most about 200 μm, at most about 100 μm, at most about 1 μm, at most about 900 nm, at most about 800 nm, at most about 700 nm, at most about 600 nm, at most about 500 nm, or less.

In some embodiments, lipid membrane delivery systems can comprise amphipathic or amphiphilic molecules such as phospholipids or combinations of phospholipids (e.g., phosphatidylcholine, phosphatidylethanolamine, phosphatidic acid, phosphatidylserine, and phosphoinositides). In some embodiments, lipid membrane delivery systems can contain additive(s) such as sterols, polyethylene glycol, cholesterol, dicethylphosphate, stearyl amine, etc. In some embodiments, the lipid membrane delivery system content can be adjusted to achieve a sub-antimicrobial concentration of defensins. In some embodiments, unilamellar vesicles/liposomes can be produced using high shear techniques. In some cases, the unilamellar vesicles/liposome can have a greater Zeta Potential than the typical liposome and can allow for smaller/more uniform particle size with increased stability. Zeta Potential is an indicator of the electronic charge on the surface of any macroscopic material that is in contact with a liquid. In some embodiments, Zeta Potential can be used to predict and control the stability of suspensions. In some cases, a higher Zeta Potential can be associated with greater the stability of the molecule because the charged particles are able to repel and overcome their innate affinity to assemble.

In some embodiments, the defensins can have a profound effect on stem cell activity in dermal and hypodermal layers. In some embodiments, the liposomal formulations can have the ability to transport the defensins to invade the hair follicle and/or skin pores, and/or stratum corneum, and/or penetrate through nail plate to a depth and concentration sufficient to activate LGR6+ cells. In some embodiments, the use of lipid membrane delivery systems can aid in the delivery of defensins.

In one aspect, the composition can include additional ingredients that nourish and support the recruited stem cells in healthy nails and lips. Non-limiting examples of additional ingredients include albumin (e.g., human serum albumin, bovine serum albumin), egg albumin (albumen), recombinant albumin, plant hydrolysate, and P-cyclodextrin, glutamine, phospholipids (liposomes), fibronectin, hyaluronate, plant hydrolysate, L-alanyl-L-glutamine, gelatin, Vitamin E (tocopheryl nicotinate), ubiquinone (coenzyme Q₁₀), gelatin, recombinant gelatin, hyaluronic acid, Epidermal Growth Factor, or any combinations thereof. In some embodiments, the additional ingredient can be albumin (e.g., human serum albumin, bovine serum albumin). In some embodiments, the additional ingredient can be egg albumin (albumen). In some embodiments, the additional ingredient can be recombinant albumin. In some embodiments, the additional ingredient can be plant hydrolysate. In some embodiments, the additional ingredient can be P-cyclodextrin. In some embodiments, the additional ingredient can be glutamine. In some embodiments, the additional ingredient can be phospholipids. In some embodiments, the additional ingredient can be fibronectin. In some embodiments, the additional ingredient can be hyaluronate. In some embodiments, the additional ingredient can be plant hydrolysate. In some embodiments, the additional ingredient can be L-alanyl-L-glutamine. In some embodiments, the additional ingredient can be gelatin. In some embodiments, the additional ingredient can be Vitamin E (tocopheryl nicotinate). In some embodiments, the additional ingredient can be ubiquinone (coenzyme Q₁₀). In some embodiments, the additional ingredient can be gelatin. In some embodiments, the additional ingredient can be recombinant gelatin. In some embodiments, the additional ingredient can be hyaluronic acid. In some embodiments, the additional ingredient can be Epidermal Growth Factor.

### Lip and Nail Formulations and Methods

One aspect of the present disclosure provides compositions and methods of use of a topical formulation as herein disclosed. In some embodiments, the topical formulation can be formulated for use on a lip or nail. In some embodiments, the topical formulation can be formulated for use on a lip or skin around the lip. In some embodiments, the topical formulation can be formulated for use on a nail. In some embodiments, the composition can be applied on broken, damaged or removed nail. In some embodiments, the composition can be applied on damaged or wounded lips.

In some embodiments, the method can comprise providing a topical formulation comprising a first defensin and a cosmetically acceptable ingredient as described herein. In some embodiments, the method can further comprise applying the topical formulation to the lip or nail of the individual. In some embodiments, the topical formulation can alleviate the cosmetic concern of the lip or nail of the individual.

In some embodiments, the topical formulation can be applied to the lip. In some cases, the topical formulation can alleviate the cosmetic concern of the lip. In some cases, the cosmetic concern of the lip can be wrinkles on the lip, wrinkles around contour of the lip, an appearance of aging of the lip, discoloration of the lip, dryness of the lip, or any combinations thereof. In some cases, the cosmetic concern of the lip can be wrinkles on the lip. In some cases, the cosmetic concern of the lip can be wrinkles around contour of the lip. In some cases, the cosmetic concern of the lips can the appearance of aging of the lip. In some cases, the cosmetic concern of the lip can be discoloration of the lip. In some cases, the cosmetic concern of the lip can be dryness of the lip.

In some embodiments, the topical formulation can be applied to the nail in some embodiments, the topical formulation can be applied to nail root, eponychium, proximal nail fold, lanula, cuticle, lateral nail fold, nail body/plate and hyponychium. In some cases, the topical formulation can alleviate the cosmetic concern of the nail of an individual. In some embodiments, the cosmetic concern of the nail can be brittle nail, thin nail, weakness of nail, nail discoloration, slow nail growth, or any combinations thereof. In some cases, the cosmetic concern of the nail can be brittle nail. In some cases, the cosmetic concern of the nail can be thin nail. In some cases, the cosmetic concern of the nail can be weakness of nail. In some cases, the cosmetic concern of the nail can be nail discoloration. In some cases, the cosmetic concern of the nail can be slow nail growth.

### Kits

Any topical formulation as described herein can be packaged in a kit. A kit can comprise an applicator brush. An applicator brush can comprise a button on the bottom of the brush which, when pushed, dispenses serum onto the brush.

### Embodiments

Embodiment 1. A composition comprising a topical formulation that comprises:
a) a first defensin; and
b) a cosmetically acceptable ingredient,
   wherein the topical formulation is formulated for use on a lip or a nail.

Embodiment 2. The composition of embodiment 1, wherein the topical formulation is formulated for use on the lip, and wherein the topical formulation is capable of alleviating a cosmetic concern of the lip.

Embodiment 3. The composition of embodiment 2, wherein the cosmetic concern of the lip comprises wrinkles on the lip, wrinkles around contour of the lip, an appearance of aging of the lip, discoloration of the lip, dryness of the lip, or any combinations thereof.

Embodiment 4. The composition of embodiment 1, wherein the topical formulation is formulated for use on the nail, and wherein the topical formulation is capable of alleviating a cosmetic concern of the nail or stimulating a regeneration of the nail.

Embodiment 5. The composition of embodiment 4, wherein the nail comprises a nail portion selected from the group consisting of nail root, eponychium, proximal nail fold, lanula, cuticle, lateral nail fold, nail body/plate and hyponychium.

Embodiment 6. The composition of embodiment 4, wherein the cosmetic concern of the nail comprises brittle nail, thin nail, weakness of nail, nail discoloration, slow nail growth, nail surface irregularities, nail surface smoothness, nail vertical ridges, nail horizontal ridges, nail hydration, cuticle hydration, aesthetic appearance of the nail, aesthetic appearance of the cuticle, or any combinations thereof.

Embodiment 7. The composition of any of embodiments 1-6, wherein the first defensin is selected from the group consisting of alpha-defensin 1, alpha-defensin 5, alpha-defensin 6, neutrophil defensin 1, neutrophil defensin 2, neutrophil defensin 3, neutrophil defensin 4, theta-defensin, beta-defensin 1, beta-defensin 2, beta-defensin 3 and beta-defensin 4.

Embodiment 8. The composition of any of embodiments 1-7, wherein the first defensin is present in a concentration effective to recruit Lgr6+ stem cells.

Embodiment 9. The composition of any of embodiments 1-7, wherein the first defensin is present in a concentration of about 0.01 ng/ml to about 1000 ng/ml.

Embodiment 10. The composition of any of embodiments 1-7, wherein the first defensin is present in a concentration of about 0.01 ng/ml to about 100 ng/ml.

Embodiment 11. The composition of any of embodiments 1-7, wherein the first defensin is present in a concentration of about 1 ng/ml to about 100 ng/ml.

Embodiment 12. The composition of any of embodiments 1-11, wherein the topical formulation further comprises a second defensin, wherein the second defensin is selected from the group consisting of alpha-defensin 1, alpha-defensin 5, alpha-defensin 6, neutrophil defensin 1, neutrophil defensin 2, neutrophil defensin 3, neutrophil defensin 4, theta-defensin, beta-defensin 1, beta-defensin 2, beta-defensin 3 and beta-defensin 4.

Embodiment 13. The composition of embodiment 11, wherein the second defensin is present in a concentration effective to recruit Lgr6+ stem cells.

Embodiment 14. The composition of embodiment 12, wherein the second defensin is present in a concentration of about 0.01 ng/ml to about 1000 ng/ml.

Embodiment 15. The composition of embodiment 12, wherein the second defensin is present in a concentration of about 0.01 ng/ml to about 100 ng/ml.

Embodiment 16. The composition of embodiment 12, wherein the second defensin is present in a concentration of about 1 ng/ml to about 100 ng/ml.

Embodiment 17. The composition of embodiment 12, wherein the first defensin and the second defensin are present in a mass ratio of about 1:10 to about 10:1.

Embodiment 18. The composition of embodiment 12, wherein the first defensin and the second defensin are present in a mass ratio of about 1:5 to about 5:1.

Embodiment 19. The composition of any of embodiments 1-18, wherein the cosmetically acceptable ingredient is selected from the group consisting of Leuconostoc/radish root ferment filtrate, albumin, l-alanyl-l-glutamine, phospholipids, tocopheryl acetate, ubiquinone, Caesalpinia spinosa fruit extract, Helianthus annuus sprout extract, Pistacia lentiscus gum, squalane, panthenol, biotin, niacinamide, d-l methionine and any combinations thereof.

Embodiment 20. The composition of any of embodiments 1-18, wherein the cosmetically acceptable ingredient is selected from the group consisting of Leuconostoc/radish root ferment filtrate, albumin, l-alanyl-l-glutamine, phospholipids, tocopheryl acetate, ubiquinone, Undaria pinnatifida extract, Caesalpinia spinosa fruit extract, Kappaphycus alvarezii extract, ceratonia siliqua fruit extract, brassica alba sprout extract and any combinations thereof.

Embodiment 21. The composition of any of embodiments 1-18, wherein the cosmetically acceptable ingredient comprises Caesalpinia spinosa fruit extract.

Embodiment 22. A method for alleviating a cosmetic concern of a lip or a nail of an individual, comprising: providing a topical formulation comprising a first defensin and a cosmetically acceptable ingredient, applying the topical formulation to the lip or the nail of the individual, wherein the topical formulation alleviates the cosmetic concern of the lip or the nail of the individual.

Embodiment 23. The method of embodiment 22, wherein the topical formulation is applied to the lip or skin around the lip, and wherein the topical formulation alleviates the cosmetic concern of the lip.

Embodiment 24. The method of embodiment 23, wherein the cosmetic concern of the lip comprises wrinkles on the lip, wrinkles around contour of the lip, an appearance of aging of the lip, discoloration of the lip, dryness of the lip, or any combinations thereof.

Embodiment 25. The method of embodiment 22, wherein the topical formulation is applied to the nail, and wherein the topical formulation alleviates the cosmetic concern of the nail or stimulates a regeneration of the nail.

Embodiment 26. The method of embodiment 24, wherein the nail comprises a nail portion selected from the group consisting of nail root, eponychium, proximal nail fold, lanula, cuticle, lateral nail fold, nail body/plate and hyponychium.

Embodiment 27. The method of embodiment 24, wherein the cosmetic concern of the nail comprises brittle nail, thin nail, weakness of nail, nail discoloration, slow nail growth, nail surface irregularities, nail surface smoothness, nail vertical ridges, nail horizontal ridges, nail hydration, cuticle hydration, aesthetic appearance of the nail, aesthetic appearance of the cuticle, or any combinations thereof.

Embodiment 28. The method of any of embodiments 22-27, wherein the first defensin is selected from the group consisting of alpha-defensin 1, alpha-defensin 5, alpha-defensin 6, neutrophil defensin 1, neutrophil defensin 2, neutrophil defensin 3, neutrophil defensin 4, theta-defensin, beta-defensin 1, beta-defensin 2, beta-defensin 3 and beta-defensin 4.

Embodiment 29. The method of any of embodiments 22-28, wherein the first defensin is present in a concentration effective to recruit Lgr6+ stem cells.

Embodiment 30. The method of any of embodiments 22-28, wherein the first defensin is present in a concentration of about 0.01 ng/ml to about 1000 ng/ml.

Embodiment 31. The method of any of embodiments 22-28, wherein the first defensin is present in a concentration of about 0.01 ng/ml to about 100 ng/ml.

Embodiment 32. The method of any of embodiments 22-28, wherein the first defensin is present in a concentration of about 1 ng/ml to about 100 ng/ml.

Embodiment 33. The method of any of embodiments 22-32, wherein the topical formulation further comprises a second defensin, wherein the second defensin is selected from the group consisting of alpha-defensin 1, alpha-defensin 5, alpha-defensin 6, neutrophil defensin 1, neutrophil defensin 2, neutrophil defensin 3, neutrophil defensin 4, theta-defensin, beta-defensin 1, beta-defensin 2, beta-defensin 3 and beta-defensin 4.

Embodiment 34. The method of embodiment 33, wherein the second defensin is present in a concentration effective to recruit Lgr6+ stem cells.

Embodiment 35. The method of embodiment 33, wherein the second defensin is present in a concentration of about 0.01 ng/ml to about 1000 ng/ml.

Embodiment 36. The method of embodiment 33, wherein the second defensin is present in a concentration of about 0.01 ng/ml to about 100 ng/ml.

Embodiment 37. The method of embodiment 33, wherein the second defensin is present in a concentration of about 1 ng/ml to about 100 ng/ml.

Embodiment 38. The method of embodiment 33, wherein the first defensin and the second defensin are present in a mass ratio of about 1:10 to about 10:1.

Embodiment 39. The method of embodiment 33, wherein the first defensin and the second defensin are present in a mass ratio of about 1:5 to about 5:1.

Embodiment 40. The method of any of embodiments 22-39, wherein the cosmetically acceptable ingredient is selected from the group consisting of Leuconostoc/radish root ferment filtrate, albumin, l-alanyl-l-glutamine, phospholipids, tocopheryl acetate, ubiquinone, Caesalpinia spinosa fruit extract, Helianthus annuus sprout extract, Pistacia lentiscus gum, squalane, panthenol, biotin, niacinamide, d-l methionine and any combinations thereof.

Embodiment 41. The method of any of embodiments 22-39, wherein the cosmetically acceptable ingredient is selected from the group consisting of Leuconostoc/radish root ferment filtrate, albumin, l-alanyl-l-glutamine, phospholipids, tocopheryl acetate, ubiquinone, Undaria pinnatifida extract, Caesalpinia spinosa fruit extract, Kappaphycus alvarezii extract, ceratonia siliqua fruit extract, brassica alba sprout extract and any combinations thereof.

Embodiment 42. The method of any of embodiments 22-39, wherein the cosmetically acceptable ingredient comprises Caesalpinia spinosa fruit extract.

### EXAMPLES

While the cosmetic formulations may be prepared using any number of ingredients and formulations known in the art, preferred topical formulations include those that are ready-to-use and can be applied by a user. Therefore, with respect to cosmetically acceptable carriers, all cosmetically acceptable carriers are contemplated and include creams, oil-in-water emulsions, water-in-oil emulsions, foams, mousses, ointments, lotions, suspensions, serum, gels, etc.

### Example 1: Lip Formulation Testing

### Exemplary Topical Cosmetic Lip Formulation Ingredient List 1:

Leuconostoc/Radish Root Ferment Filtrate, Albumin, l-Alanyl-l-Glutamine, Phospholipids, Tocopheryl Acetate, Ubiquinone, Undaria Pinnatifida extract, Caesalpinia spinosa Fruit Extract, Kappaphycus alvarezii Extract, Ceratonia siliqua (Carob) Fruit Extract, Brassica alba Sprout Extract.

### Exemplary Topical Cosmetic Lip Formulation Ingredient List 2:

Caprylic/Capric Triglyceride, Water, Ricinus communis (Castor) Seed Oil, Bis-Diglyceryl Polyacryladipate-2, Hydrogenated Rapeseed Oil, Ethylhexyl Palmitate, Butyrospermum parkii (Shea) Butter, Stearalkonium Hectorite, Pyrus malus (Apple) Fruit Extract, Glycerin, Niacinamide, Sambucus nigra Fruit Extract, Lauroyl Lysine, Glycine soja (Soybean) Seed Extract, Propylene Carbonate, Phospholipids.

1.0% or less: Alpha-Defensin 5 (sh-Oligopeptide-109 SP), Beta-Defensin 3 (sh-Oligopeptide-108 SP), Ubiquinone, Tocopheryl Nicotinate, Tetrahexyldecyl Ascorbate, Sodium Hyaluronate, Undaria pinnatifida Extract, Palmitoyl Tripeptide-1, Brassica alba Sprout Extract, Epigallocatechin Gallatyl Glucoside, Caesalpinia spinosa Fruit Extract, Kappaphycus alvarezii Extract, Spirodela polyrhiza Extract, Bisabolol, Panthenol, Tocopheryl Acetate, Glycyrrhiza glabra (Licorice) Leaf Extract, Citrus limon (Lemon) Peel Extract, Albumin, l-Alanyl-l-Glutamine, Polyglyceryl-2 Dipolyhydroxystearate, Glucomannan, Trihydroxystearin, Tribehenin, Sorbitan Isostearate, Potassium Sorbate, Leuconostoc/Radish Root Ferment Filtrate, Sodium Benzoate, Citric Acid, Sodium Chloride.

12.5% Age Repair Defensins 5× Liposome

### Lip Hydration

A number of volunteers with dry lips are included in the study to test the immediate hydration effect of the composition on lips. Before application of the composition, the cutaneous hydration of the lips of each volunteer is measured using a corneometer. A defined quantity of the composition is applied in a standardized way by a technician on the lips of the volunteers. Two hours after application of the composition on the lips, the cutaneous hydration of the lips is measured again to test for an increase in lip hydration after a single application

### Wrinkles Around Contours of Lip

A number of volunteers with visible wrinkles around the contours of lips are included in the study. On day 0 of the study before application of the composition, 3D acquisitions of wrinkles around the contour of the lips are generated by fringe projection and volunteers fill out a self-evaluation of wrinkles using a mirror at the test center. Following initial acquisitions and evaluations, volunteers apply the composition to their lips. One hour after application, volunteers fill out a second self-evaluation of the wrinkles using a mirror and undergo a second 3D acquisition by fringe projection. The volunteers then return home with the composition and apply the composition once daily from day 1 to day 6 of the study design. On day 7, volunteers return to the test center to complete a third self-evaluation using a mirror and a third 3D acquisition by fringe projection. The three self-evaluations and three 3D acquisitions per volunteer are compared to see if wrinkles around the contours of the lips became less visible over the course of the study.

### Example 2: Nail Formulation Testing

### Example Topical Cosmetic Nail Formulation Ingredient List:

Leuconostoc/Radish Root Ferment Filtrate, Albumin, l-Alanyl-l-Glutamine, Phospholipids, Tocopheryl Acetate, Ubiquinone, Caesalpinia spinosa Fruit Pod Extract/Caesalpinia spinosa Fruit Extract, Helianthus annuus (Sunflower) Sprout Extract, Pistacia lentiscus gum, Squalane, Panthenol, Biotin, Niacinamide, D-L Methionine.

### Nail Thickness

A number of volunteers with weak nails are included in this study. The volunteers are instructed to not wear nail polish during the duration of the study. On day 0 of the study, the nail thickness of the volunteer's nails is measured using a high frequency echography machine (e.g., DermaScan CR 2D) at the test center. The volunteers then apply the composition to their nails. The volunteers are sent home with the composition and instructed to apply the composition once a day on days 1-27 of the study. On day 28, the volunteers return to the test center to measure the thickness of their nails using the high frequency echography machine.

### Example 3: Improvements in Nail Characteristics

### Study Design

Four subjects (3 females and 1 male) were included in a study to determine the efficiency of a nail composition of the present disclosure. Prior to the start of the study, the baseline characteristics of the subject's nails were measured. The nail characteristics examined in this study included: horizontal and vertical ridges of the nail, surface smoothness of the nail, nail plate hardness, nail free edge thickness, nail free edge length measurement, and skin hydration.

The nail composition was applied to the subject's nails and the skin all around the nails on both hands twice a day. The subjects applied the nail composition twice a day every day for 91 days, with nail characteristics measured on day 0 (baseline characteristics), day 28, day 28, day 56, and day 91. The nail free edge was cut to control the length of the nail on day 14, day 42, and day 77 of the study. In addition, high resolution images of the subject's nail were taken on day 0, day 28, day 56, and day 91 of the study.

### Horizontal and Vertical Ridges of Nails Nail Smoothness

The depth and depth of horizontal and vertical ridges of the subject's nails were measured using the Visioscan® imaging system. The Visioscan® imaging system has a measuring heading with a CCD-camera and two metal halogen lamps positioned opposite of each other in order to ensure even illumination of the measuring filed on the nail. The grey level distribution of the pixels in the image corresponded to different phenomena, such as dark pixels representing ridges on the nail. Following imaging on the subject's nails, the Visioscan® software automatically calculated the horizontal and vertical ridges (e.g., roughness) of the nail.

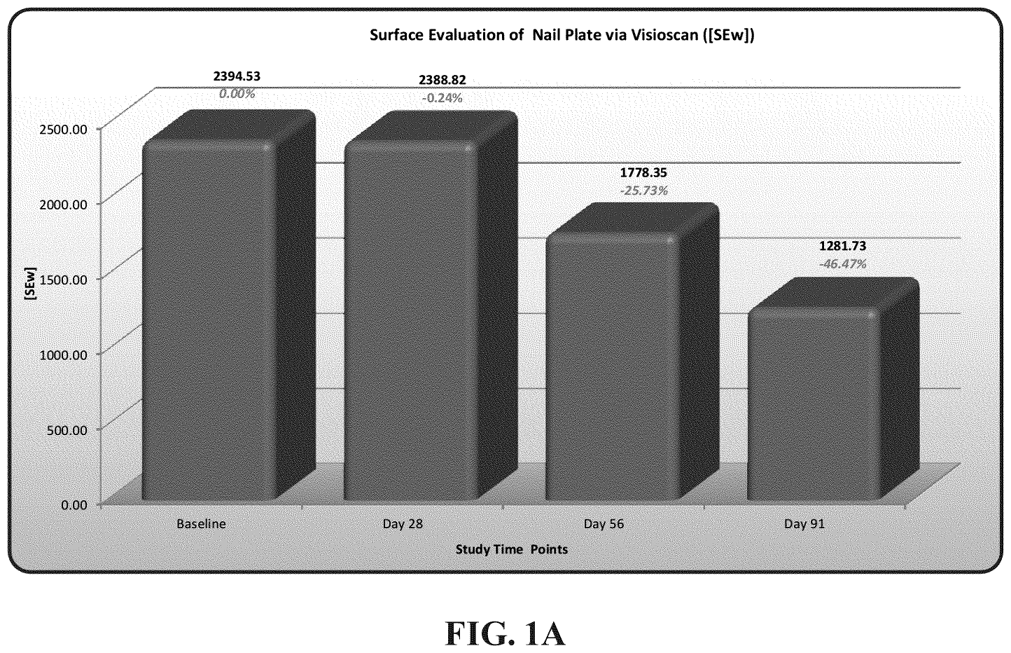

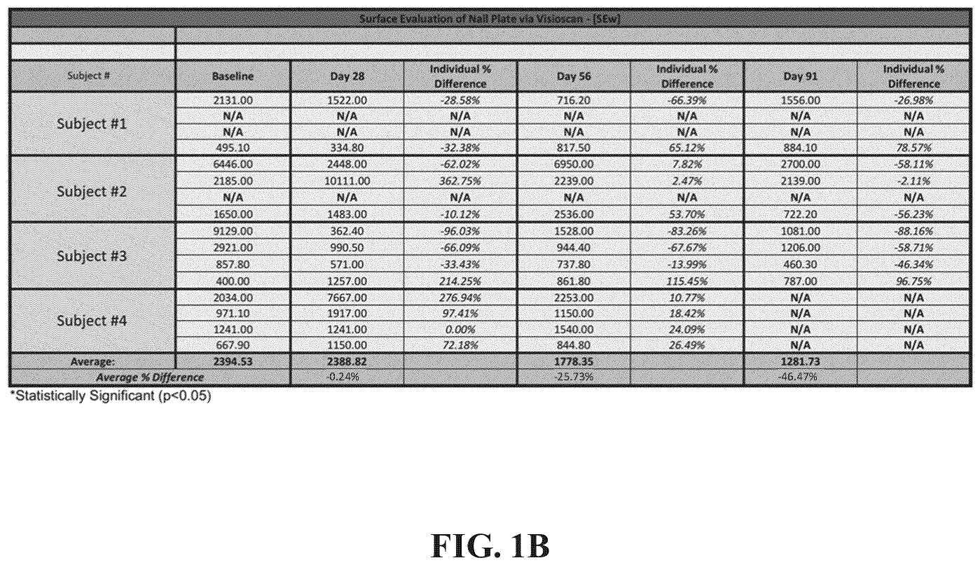

The nail composition was shown to reduce the horizontal and vertical ridges on the nail over the course of the study has shown in FIGS. 1A-1B, with an average reduction in ridges of about 25% on day 56 and about 46% on day 91 of the study.

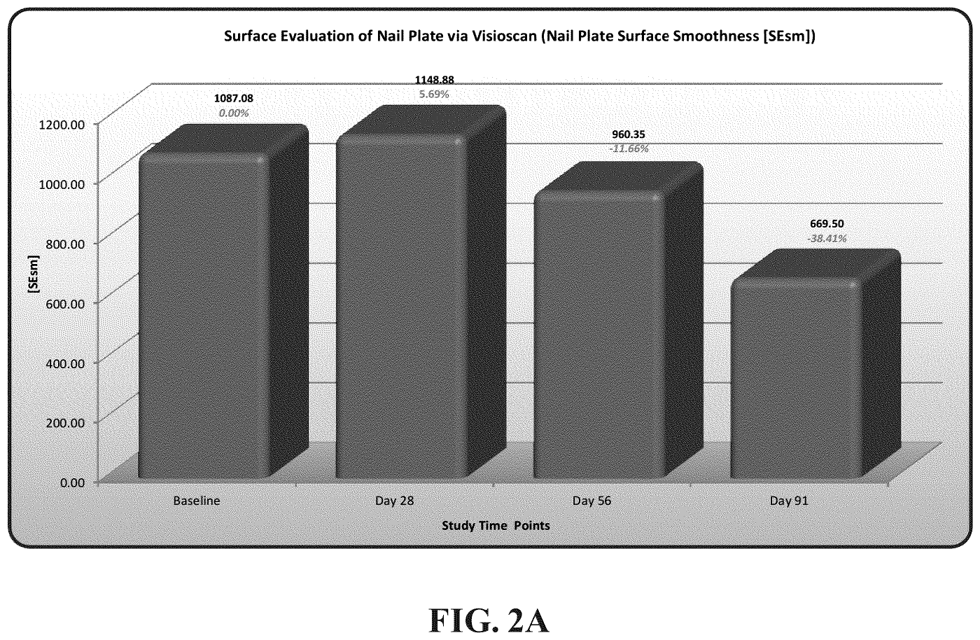

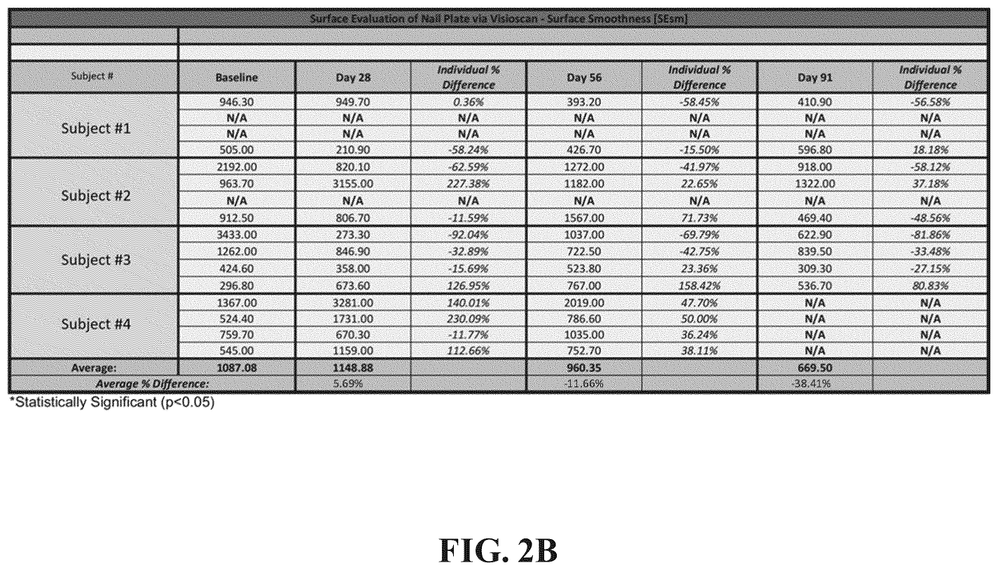

In addition, the Visioscan® calculations of the average depth and width of the ridges on the nail also showed that the nail composition improved the overall smoothness of the nail, as shown in FIGS. 2A-2B. Overall, the nail composition resulted in an average reduction of ridges in the nail of about 11% on day 56 and about 38% on day 91 of the study.

### Nail Plate Hardness

The Nail Plate hardness of the subject's nails were measured using a durometer. The durometer measured the resistance force of the penetration of a pin into the nail under a known spring load; the amount of penetration (e.g., 2.5 millimeter maximum) of the pin was converted into a hardness reading on a scale with 100 units maximum.

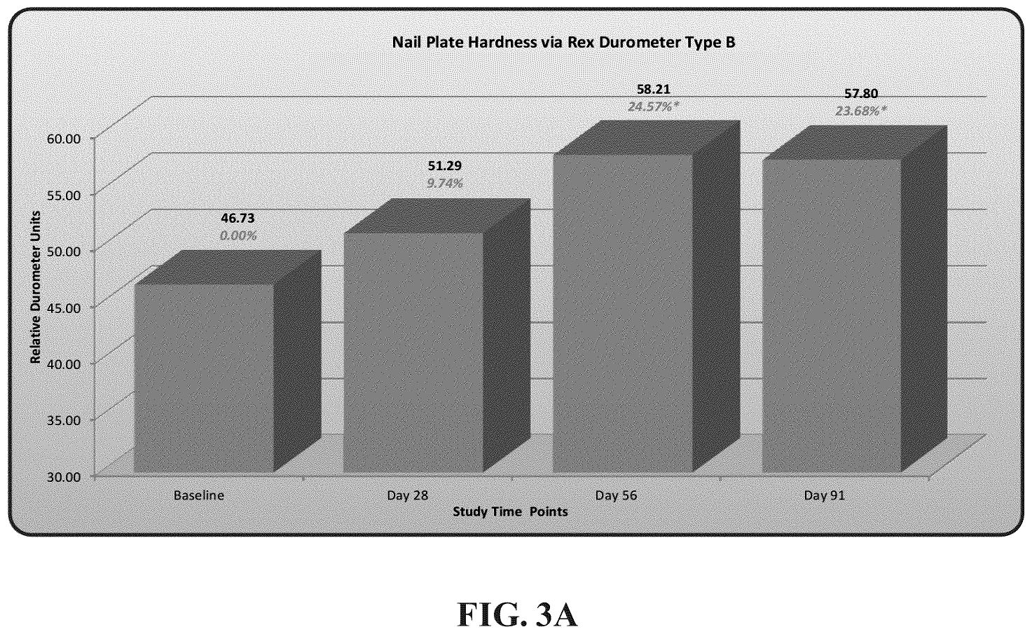

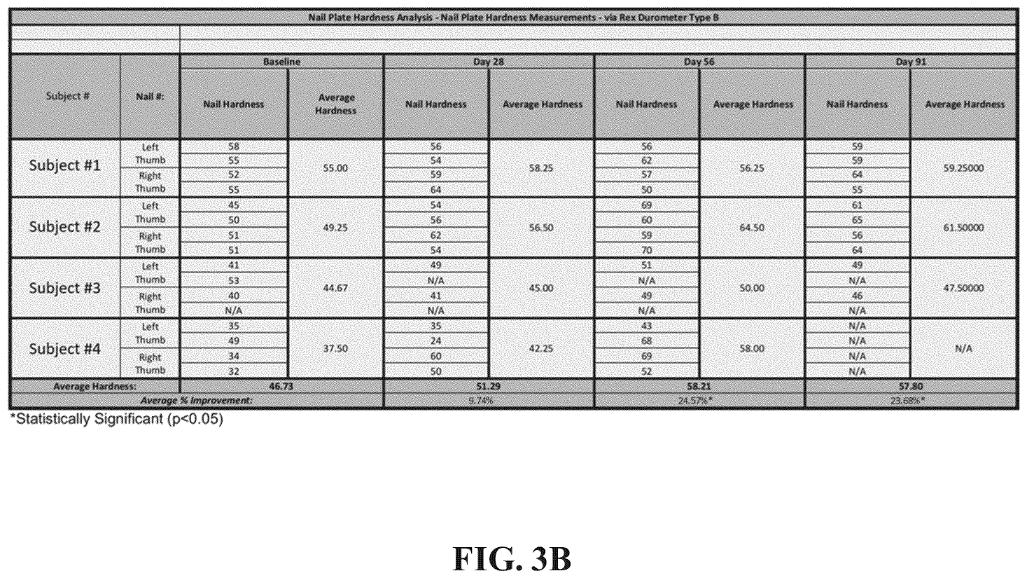

The nail composition was shown to increase the average nail plate hardness over the course of the study, with the nail plate being significantly harder on day 56 and day 91 of the study, as shown in FIGS. 3A-3B.

### Nail Free Edge Thickness

The nail free edge thickness was measured by cutting the nail free edge on the thumb (both right and left hand) and measuring the thickness of the cut nail free edge of optical micrometry on day 0, day 28, day 59, and day 91 of the study.

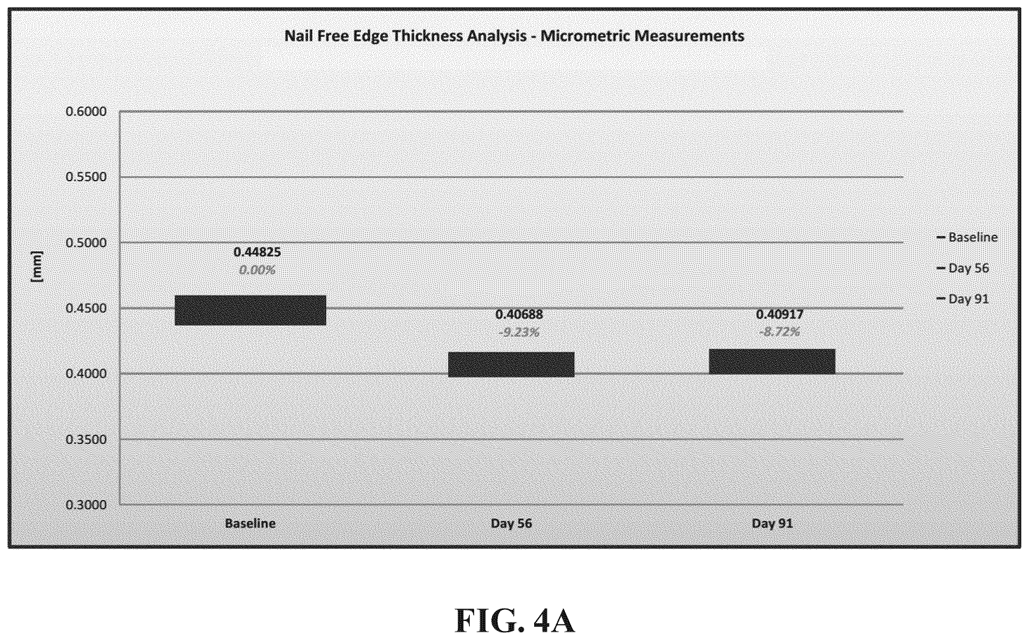

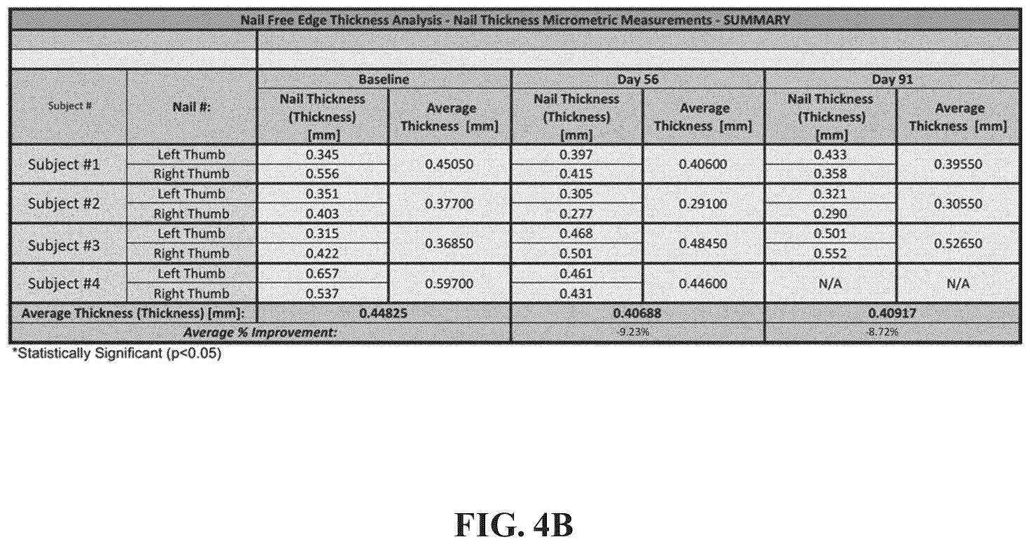

The nail composition was shown to reduce the average thickness of the nail free edge on the thumb nail by an average of about 9% on day 56 and day 91 of the study, as shown in FIGS. 4A-B below.

### Nail Free Edge Length

The length of the nail free edge length was measured by high resolution matched scientific photography. Each nail of the subject on each hand was photographed at the following time points: day 0 (baseline), day 28, day 56, and day 91. The images taken at each time point were then analyzed by PhotoGrammetrix® Image Analysis to calculate the length of the nail free edge length.

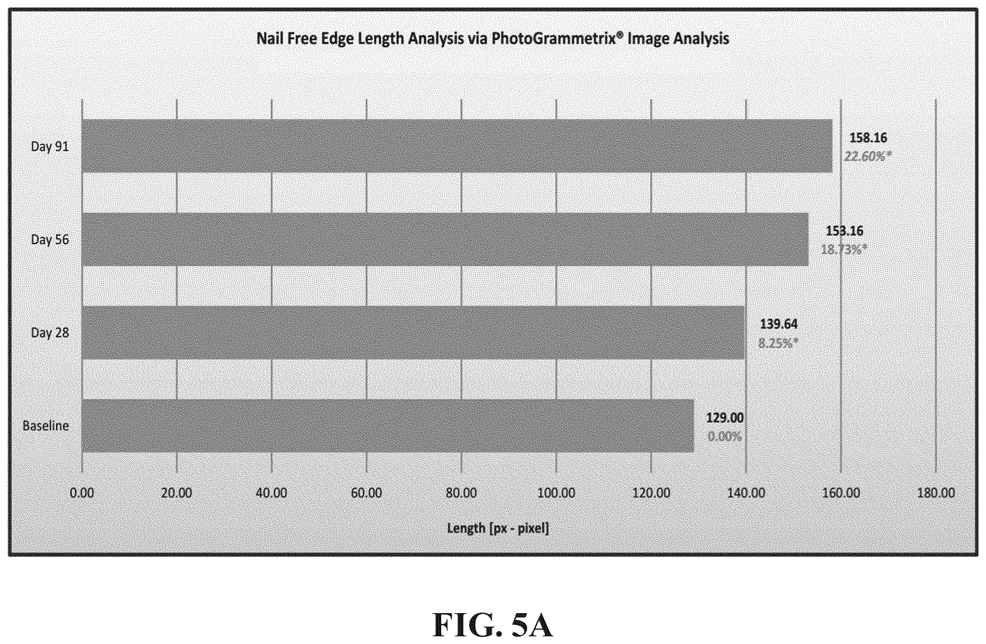

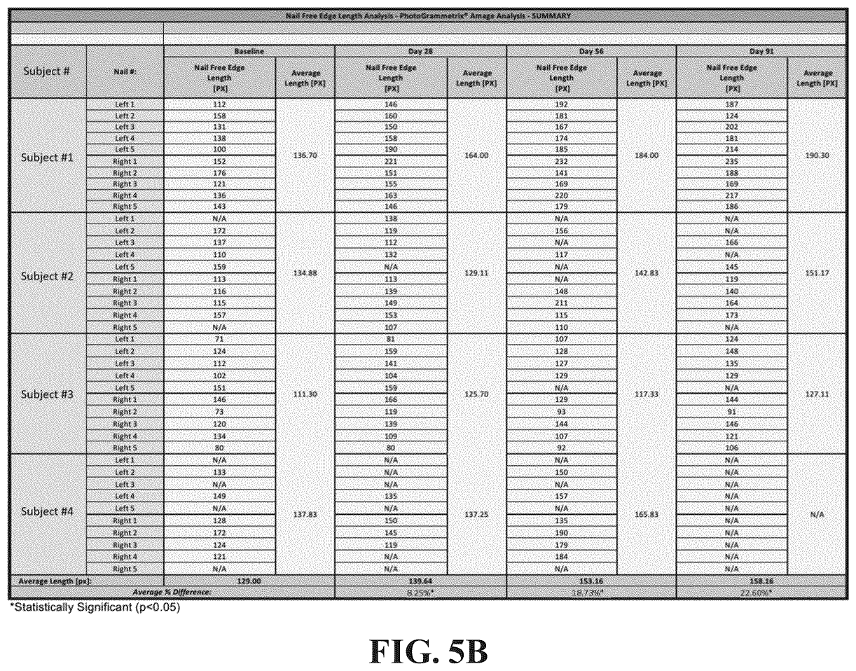

The nail composition was shown to significantly increase the average nail free edge length at day 28, day 56, and day 91 of the study has shown in FIGS. 5A-5B.

### Skin Hydration

The hydration of the skin around the nail (e.g., nail root) was measured via a nova dermal phase meter. The nova dermal phase meter measured skin surface impedance to determine the electroconductivity of the skin around the nail. The measurements obtained were a relative measurement of the retained water content of the skin as a function of the skin's dielectric value. The skin impedance measurement was recorded automatically by the nova dermal phase meter once equilibrium was achieved.

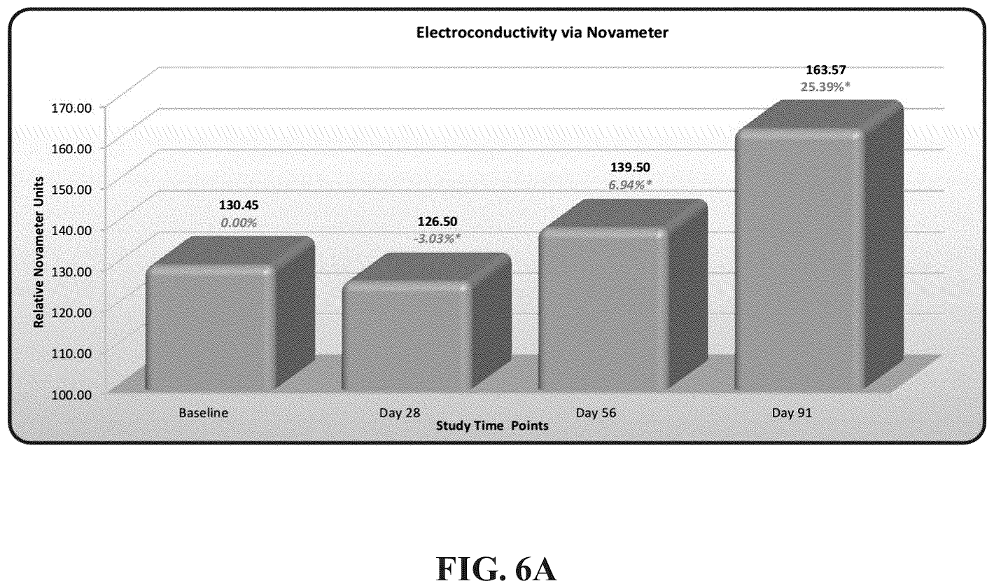

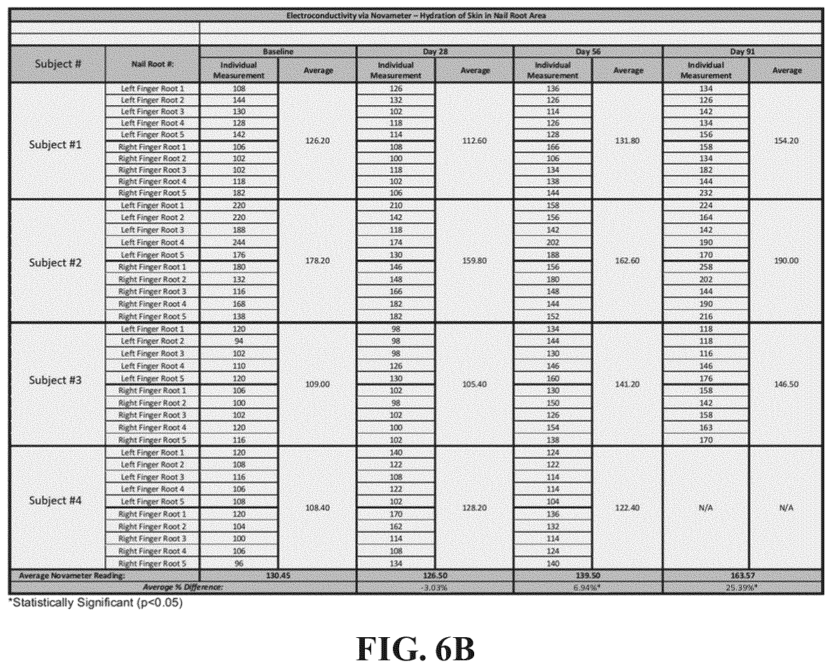

The nail composition was shown to significantly increase the average level of skin moisture in the skin around the nail on day 56 and day 91 of the study, as shown in FIGS. 6A-6B.

### Conclusions

Overall, application of a nail composition of the present disclosure resulted in improvements in overall nail surface smoothness, nail plate hardness, nail free edge thickness and length, and hydration of skin around the nail.

### Example 4: Analysis of Lip Characteristics Following Lip Composition Use

Subjects are included in a study to determine the efficiency of a lip composition of the present disclosure. Prior to the start of the study, baseline characteristics of the subject's lips are measured. This study examines the following lip characteristics: dry lips, trans-epidermal water loss of lips, lip plumping, lip color, and wrinkles on lip line.

The lip composition is applied to the subject's lip and skin around the lip twice a day. The subjects applied the nail composition twice a day every day for 91 days, with lips characteristics evaluated on day 0 (baseline characteristics), day 28, day 56, and day 91.

### Dry Lips

The dryness of the subject's lips is determined by taking photographs of the subject's lips on day 0, day 28, day 56, and day 91 of the study. The images of the subject's lips are graded on the scale as shown in Table 1. At the conclusion of the study, the lip dryness grades from each timepoint are analyzed to determine if there is a decrease in lip dryness by using the lip composition.

TABLE 1
Lip Dryness Characteristics and Lip Dryness Grading Score

| Characteristics of Subject's Lips | Dryness Score |
|---|---|
| No dryness or chapping evident | 0 |
| Slight, but definite roughness; fine scaling | 1-2 |
| Moderate roughness; coarse scaling; slight cracking | 3-4 |
| Marked roughness; coarse scaling; obvious cracking | 5-6 |
| Very marked roughness; course scaling; cracking | 7-8 |
| progressing to fissuring | |

### Lip Hydration Trans-Epidermal Water Loss Assay

The trans-epidermal water loss (TEWL) of the subject's lips is measured using a vapometer. To measure TEWL, the vapometer is placed directly on the lips of the subject on day 0, day 28, day 56, and day 91 of the study. Once placed on the lips of the subject, the vapometer automatically calculates the TWEL of the subject's lips in units of grams per meter² per hour (g/m²/h). At the conclusion of the study, the TWEL values calculated by the vapometer at each timepoint are compared to determine if there is a decrease in the TWEL of the lips by using the lip composition.

### Lip Plumping

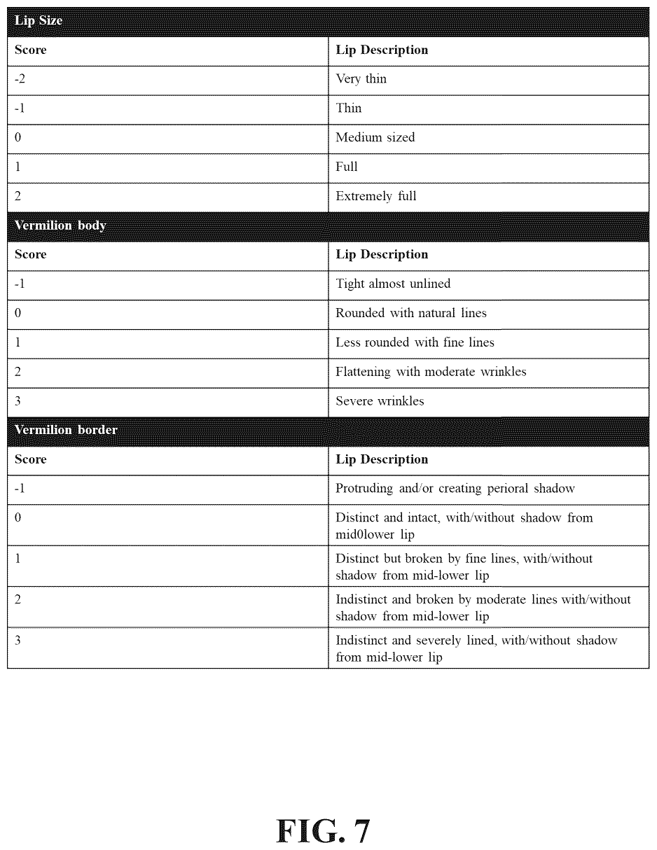

The fullness (e.g., plumpness) of the subject's lips is determined by taking photographs of the subject's lips on day 0, day 28, day 56, and day 91 of the study. The images of the subject's lips are graded on the photometric scale as shown in FIG. 7. At the conclusion of the study, the lip fullness grades from each timepoint are analyzed to determine if there is an increase lip fullness by using the lip composition.

### Lip Color

The color of the subject's lips is determined by taking photographs of the subject's lips on day 0, day 28, day 56, and day 91 of the study. The photographs are analyzed for appearance of lip color (e.g., pink, red, pale, purple) and evenness of lip color (e.g., presence of spots on the lips, spotted lips). Healthy pinks are considered to be pink or light red lips. At the conclusion of the study, the images of the lips are compared to determine if there is an increase in the number of subject's with pink or light red lips.

### Wrinkles at Lip Line

The evaluation of wrinkles at the lip line (e.g., lipstick wrinkles) of the subject's lips is evaluated by taking photographs of the subject's lips on day 0, day 28, day 56, and day 91 of the study. The photographs of the subject's lips are then analyzed by an image-analysis software to calculate a visual grading score of the wrinkles at the lip line of the subject. At the conclusion of the study, the scores of the wrinkles at the lip line at each timepoint are compared to determine if there is a decrease in the presence of wrinkles at the lip line by using the lip composition.

### Example 4: Application of Nail Serum Composition

A subject applies a nail root serum of a formulation as described herein twice daily to nails. The application occurs once in the morning and once in the evening.

Application is done by using an applicator with a built-in brush. One to two pumps of the serum are applied to all nails and to the skin around the nails on both hands using the built-in applicator brush. A pump of the serum is released from the formulation packaging by pushing a button on the bottom of the brush and distributing the serum across all nails and the skin around the nails. The serum is brushed from the bed of the nail toward the end of the nail. The serum is applied in an even distribution to the skin surrounding all sides of the nail.

The serum is left on the nails and the skin around the nails for at least 30 minutes. In some experimental procedures, the serum is left on the nails and the skin around the nails overnight.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for alleviating a cosmetic concern of a lip or a nail of an individual, comprising:
providing a topical formulation comprising a first defensin and a cosmetically acceptable ingredient,
applying the topical formulation to the lip or the nail of the individual, wherein the topical formulation alleviates the cosmetic concern of the lip or the nail of the individual.

2. The method of claim 1, wherein the topical formulation is applied to the lip or skin around the lip, and wherein the topical formulation alleviates the cosmetic concern of the lip.

3. The method of claim 2, wherein the cosmetic concern of the lip comprises wrinkles on the lip, wrinkles around contour of the lip, an appearance of aging of the lip, discoloration of the lip, dryness of the lip, or any combinations thereof.

4. The method of claim 1, wherein the topical formulation is applied to the nail, and wherein the topical formulation alleviates the cosmetic concern of the nail or stimulates a regeneration of the nail.

5. The method of claim 3, wherein the nail comprises a nail portion selected from the group consisting of nail root, eponychium, proximal nail fold, lanula, cuticle, lateral nail fold, nail body/plate and hyponychium.

6. The method of claim 3, wherein the cosmetic concern of the nail comprises brittle nail, thin nail, weakness of nail, nail discoloration, slow nail growth, nail surface irregularities, nail surface smoothness, nail vertical ridges, nail horizontal ridges, nail hydration, cuticle hydration, aesthetic appearance of the nail, aesthetic appearance of the cuticle, or any combinations thereof.

7. The method of claim 1, wherein the first defensin is selected from the group consisting of alpha-defensin 1, alpha-defensin 5, alpha-defensin 6, neutrophil defensin 1, neutrophil defensin 2, neutrophil defensin 3, neutrophil defensin 4, theta-defensin, beta-defensin 1, beta-defensin 2, beta-defensin 3 and beta-defensin 4.

8. The method of claim 1, wherein the first defensin is present in a concentration effective to recruit Lgr6+ stem cells.

9. The method of claim 1, wherein the first defensin is present in a concentration of about 0.01 ng/ml to about 1000 ng/ml.

10. The method of claim 1, wherein the first defensin is present in a concentration of about 0.01 ng/ml to about 100 ng/ml.

11. The method of claim 1, wherein the first defensin is present in a concentration of about 1 ng/ml to about 100 ng/ml.

12. The method of claim 1, wherein the topical formulation further comprises a second defensin, wherein the second defensin is selected from the group consisting of alpha-defensin 1, alpha-defensin 5, alpha-defensin 6, neutrophil defensin 1, neutrophil defensin 2, neutrophil defensin 3, neutrophil defensin 4, theta-defensin, beta-defensin 1, beta-defensin 2, beta-defensin 3 and beta-defensin 4.

13. The method of claim 12, wherein the second defensin is present in a concentration effective to recruit Lgr6+ stem cells.

14. The method of claim 12, wherein the second defensin is present in a concentration of about 0.01 ng/ml to about 1000 ng/ml.

15. The method of claim 12, wherein the second defensin is present in a concentration of about 0.01 ng/ml to about 100 ng/ml.

16. The method of claim 12, wherein the first defensin and the second defensin are present in a mass ratio of about 1:10 to about 10:1.

17. The method of claim 12, wherein the first defensin and the second defensin are present in a mass ratio of about 1:5 to about 5:1.

18. The method of claim 1, wherein the cosmetically acceptable ingredient is selected from the group consisting of Leuconostoc/radish root ferment filtrate, albumin, l-alanyl-l-glutamine, phospholipids, tocopheryl acetate, ubiquinone, Caesalpinia spinosa fruit extract, Helianthus annuus sprout extract, Pistacia lentiscus gum, squalane, panthenol, biotin, niacinamide, d-l methionine, and any combinations thereof.

19. The method of claim 1, wherein the cosmetically acceptable ingredient is selected from the group consisting of Leuconostoc/radish root ferment filtrate, albumin, l-alanyl-l-glutamine, phospholipids, tocopheryl acetate, ubiquinone, Undaria pinnatifida extract, Caesalpinia spinosa fruit extract, Kappaphycus alvarezii extract, Ceratonia siliqua fruit extract, Brassica alba sprout extract, and any combinations thereof.

20. The method of claim 1, wherein the cosmetically acceptable ingredient comprises Caesalpinia spinosa fruit extract.
